(19) United States
(12) Patent Application Publication
(10) Pub. No.: US 2008/0051866 A1
(43) Pub. Date: Feb. 28, 2008

(54) Drug delivery devices and methods

(76) Inventors: Chao Chin Chen, Edison, NJ (US); Vipul Dave, Hillsborough, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/435,195

(22) Filed: May 16, 2006

Related U.S. Application Data
(63) Continuation-in-part of application No. 10/834,687, filed on Apr. 29, 2004.
Application No. 10/834,687 is a continuation-in-part of application No. 10/374,211, filed on Feb. 26, 2003.

(51) Int. Cl. A61F 2/84; A61F 2/82; A61L 2/16; A61M 29/00

(52) U.S. Cl. 623/1.11; 422/28; 606/200; 623/1.2; 623/1.34; 623/1.35; 623/1.42; 623/1.43; 623/1.49; 623/2.1

(57) ABSTRACT

A biocompatible material may be configured into any number of implantable medical devices including intraluminal stents. Polymeric materials may be utilized to fabricate any of these devices, including stents. The stents may be balloon expandable or self-expanding. The polymeric materials may include additives such as drugs or other bioactive agents as well as radiopaque agents. By preferential mechanical deformation of the polymer, the polymer chains may be oriented to achieve certain desirable performance characteristics.

### CROSS-REFERENCE TO RELATED APPLICATIONS

This patent application is a continuation-in-part of pending U.S. patent application Ser. No. 10/834,687, filed Apr. 29, 2004, which is a continuation-in-part of pending U.S. patent application continuation-in-part application of Ser. No. 10/374,211 filed Feb. 26, 2003, and is a continuation-in-part of co-pending U.S. patent application Ser. No. 11/362,491, filed Feb. 24, 2006, the contents of which are incorporated herein by reference.

### BACKGROUND OF THE INVENTION

1. Field of the Invention

The present invention relates to intraluminal polymeric stents, and more particularly to intraluminal polymeric stents formed from blends of polymers, blends of polymers and plasticizers, blends of polymers and radiopaque agents, blends of polymers, plasticizers and radiopaque agents, blends of polymers, radiopaque agents and therapeutic agents, blends of polymers, plasticizers, radiopaque agents and therapeutic agents, or any combination thereof. These polymeric stents may have a modified molecular orientation due to the application of stress.

2. Discussion of the Related Art

Currently manufactured intraluminal stents do not adequately provide sufficient tailoring of the properties of the material forming the stent to the desired mechanical behavior of the device under clinically relevant in-vivo loading conditions. Any intraluminal device should preferably exhibit certain characteristics, including maintaining vessel patency through an acute and/or chronic outward force that will help to remodel the vessel to its intended luminal diameter, preventing excessive radial recoil upon deployment, exhibiting sufficient fatigue resistance and exhibiting sufficient ductility so as to provide adequate coverage over the full range of intended expansion diameters.

Accordingly, there is a need to develop materials and the associated processes for manufacturing intraluminal stents that provide device designers with the opportunity to engineer the device to specific applications.

### SUMMARY OF THE INVENTION

The present invention overcomes the limitations of applying conventionally available materials to specific intraluminal therapeutic applications as briefly described above.

In accordance with one aspect, the present invention is directed to an implantable intraluminal medical device. The implantable intraluminal medical device comprises a structure formed from at least one polymer, and at least one therapeutic agent dispersed throughout the at least one polymer in a concentration of up to thirty percent.

In accordance with another aspect, the present invention is directed to an implantable intraluminal medical device. The implantable intraluminal medical device comprises a structure formed from a first material, and a coating layer affixed to the first material, the coating layer including at least one therapeutic agent dispersed throughout a polymeric material in a concentration of up to thirty percent.

In accordance with another aspect, the present invention is directed to an implantable intraluminal medical device. The implantable intraluminal medical device comprises a structure formed from at least one polymer, an at least one radiopaque agent dispersed throughout the at least one polymer in a concentration of up to thirty percent.

In accordance with another aspect, the present invention is directed to an implantable intraluminal medical device. The implantable intraluminal medical device comprises a structure formed from a first material, and a coating layer affixed to the first material, the coating layer including at least one radiopaque agent dispersed throughout a polymeric material in a concentration of up to thirty percent.

In accordance with another aspect, the present invention is directed to an implantable intraluminal medical device. The implantable intraluminal medical device comprises a structure formed from at least one polymer, at least one radiopaque agent, and at least one therapeutic agent dispersed throughout the at least one polymer in a concentration of up to thirty percent.

In accordance with another aspect, the present invention is directed to an implantable intraluminal medical device. The implantable intraluminal medical device comprises a structure formed from at least one polymer, at least one therapeutic agent, and at least one radiopaque agent dispersed throughout the at least one polymer in a concentration of up to thirty percent.

In accordance with another aspect, the present invention is directed to an implantable intraluminal medical device. The implantable intraluminal medical device comprises a structure formed from a first material, and a coating layer affixed to the first material, the coating layer including at least one therapeutic agent and at least one radiopaque agent dispersed throughout a polymeric material in a concentration of up to thirty percent.

In accordance with another aspect, the present invention is directed to an implantable intraluminal medical device. The implantable intraluminal medical device comprises a structure formed from a first material, and a coating layer affixed to the first material, the coating layer including at least one radiopaque agent and at least one therapeutic agent dispersed throughout a polymeric material in a concentration of up to thirty percent.

In accordance with another aspect, the present invention is directed to an implantable intraluminal medical device. The implantable intraluminal medical device comprises a structure having a proximal end and a distal end, the structure being formed from at least one polymer, and at least one therapeutic agent dispersed for elution of the at least one therapeutic agent from the at least one polymer, wherein the dispersion of the at least one therapeutic agent allows for elution of the at least one therapeutic agent to a distance of greater than about five mm proximal from the proximal end and to a distance of greater than about five mm distal from the distal end.

In accordance with another aspect, the present invention is directed to an implantable intraluminal medical device. The implantable intraluminal medical device comprises a structure having a proximal and a distal end, the structure being formed from a first material, and a coating layer affixed to the first material, the coating layer including at least one therapeutic agent distributed for elution of the at least one therapeutic agent in the at least one polymer, wherein the distribution of the at least one therapeutic agent allows for elution of the at least one therapeutic agent to a distance of greater than about five mm proximal from the proximal end and to a distance of greater than about five mm distal from the distal end.

In accordance with another aspect, the present invention is directed to an intraluminal medical device. The implantable intraluminal medical device comprises a structure being formed from at least one polymer, and at least one therapeutic agent distributed for elution of the at least one therapeutic agent in the at least one polymer, wherein the distribution of the at least one therapeutic agent allows for regional delivery.

In accordance with another aspect, the present invention is directed to an implantable intraluminal medical device. The implantable intraluminal medical device comprises a structure being formed from at least one polymer, and at least one therapeutic agent dispersed throughout the at least one polymer, wherein concentration of the dispersion provides for the controlled elution of the at least one therapeutic agent for greater than about one day.

In accordance with another aspect, the present invention is directed to an implantable intraluminal medical device. The implantable intraluminal medical device comprises a structure being formed from at least one polymer, and at least one therapeutic agent dispersed throughout the at least one polymer, wherein concentration of the dispersion provides for the controlled elution of the at least one therapeutic agent for greater than about sixty days.

In accordance with another aspect, the present invention is directed to an implantable intraluminal medical device. The implantable intraluminal medical device comprises a non-fibrous structure formed from at least one polymer, and at least one therapeutic agent dispersed throughout the at least one polymer in a concentration of up to thirty percent.

In accordance with another aspect, the present invention is directed to a method for forming an implantable medical device. The method comprises the steps of creating a matrix from at least one biocompatible polymer, dispersing at least one therapeutic agent in the matrix to create a raw material, the therapeutic agent having a degradation temperature, heating the raw material to a maximum solvent processing temperature in the range from about one degree Celsius less than the degradation temperature to about eighty degrees Celsius less than the degradation temperature of the at least one therapeutic agent, and forming the heated raw material into an implantable medical device.

In accordance with another aspect, the present invention is directed to a method for forming an implantable medical device. The method comprises the steps of creating a matrix from at least one biocompatible polymer, dispersing at least one therapeutic agent in the matrix to create a raw material, the therapeutic agent having a degradation temperature, heating the raw material to a maximum melt processing temperature in the range from about one degree Celsius less than the degradation temperature to about sixty degrees Celsius less than the degradation temperature of the at least one therapeutic agent, and forming the heated raw material into an implantable medical device.

In accordance with another aspect, the present invention is directed to a method for forming an implantable medical device. The method comprises the steps of forming a raw material comprising at least one polymer into a medical device having a plurality of sections, and dispersing at least one therapeutic agent into one or more of the plurality of sections to create predetermined elution profiles.

In accordance with another aspect, the present invention is directed to an implantable intraluminal medical device. The implantable intraluminal medical device comprises a structure formed from at least one polymer, the at least one polymer configured to degrade for a period in the range from about one day to about three years, and at least one therapeutic agent dispersed throughout the at least one polymer.

In accordance with another aspect, the present invention is directed to an implantable intraluminal medical device. The implantable intraluminal medical device comprises a structure formed from a first material, and a coating layer affixed to the first material, the coating layer including at least one therapeutic agent dispersed throughout a polymeric material being configured to degrade for a period in the range from about one day to about three years.

In accordance with another aspect, the present invention is directed to an implantable intraluminal medical device. The implantable intraluminal medical device comprises a structure formed from at least one polymer, the at least one polymer configured to degrade for a period in the range from about one day to about three years, and at least one radiopaque agent dispersed throughout the at least one polymer.

In accordance with another aspect, the present invention is directed to an implantable intraluminal medical device. The implantable intraluminal medical device comprises a structure formed from a first material, and a coating layer affixed to the first material, the coating layer including at least one radiopaque agent dispersed throughout a polymeric material being configured to degrade, for a period in the range from about one day to three years.

In accordance with another aspect, the present invention is directed to a method for forming an implantable medical device. The method comprises the steps of forming a raw material comprising at least one polymer into a medical device having a plurality of sections; and dispersing at least one radiopaque agent into one or more of the plurality of sections to create predetermined marker bands.

In accordance with another aspect, the present invention is directed to a method for forming an implantable medical device. The method comprises the steps of forming a raw material comprising at least one polymer into a medical device having a plurality of sections, dispersing at least one therapeutic agent into one or more of the plurality of sections to create predetermined elution profiles, and dispersing at least one radiopaque agent into one or more of the plurality of sections to create predetermined marker bands.

In accordance with another aspect, the present invention is directed to an implantable intraluminal medical device. The implantable intraluminal medical device comprises a balloon expandable structure formed from at least one polymer, and at least one therapeutic agent dispersed throughout the at least one polymer in a concentration of up to thirty percent.

In accordance with another aspect, the present invention is directed to an implantable intraluminal medical device. The implantable intraluminal medical device comprises a balloon expandable structure formed from at least one polymer, and at least one radiopaque agent dispersed throughout the at least one polymer in a concentration of up to thirty percent.

In accordance with another aspect, the present invention is directed to an intraluminal medical device. The implantable intraluminal medical device comprises a self expanding structure formed from at least one polymer, and at least one therapeutic agent dispersed throughout the at least one polymer in a concentration of up to thirty percent.

In accordance with another aspect, the present invention is directed to an implantable intraluminal medical device. The implantable intraluminal medical device comprises a self expanding structure formed from at least one polymer; and at least one radiopaque agent dispersed throughout the at least one polymer in a concentration of up to thirty percent.

In accordance with another aspect, the present invention is directed to an implantable intraluminal medical device. The implantable intraluminal medical device comprises a balloon expandable structure formed from at least one polymer, at least one therapeutic agent dispersed throughout the at least one polymer in a concentration of up to thirty percent, and at least one therapeutic agent dispersed throughout the at least one polymer in a concentration of up to thirty percent.

In accordance with another aspect, the present invention is directed to an implantable intraluminal medical device. The implantable intraluminal medical device comprises a self-expanding structure formed from at least one polymer; at least one therapeutic agent dispersed throughout the at least one polymer in a concentration of up to thirty percent and at least one radiopaque agent dispersed throughout the at least one polymer in a concentration of up to thirty percent.

In accordance with another aspect, the present invention is directed to an implantable intraluminal medical device. The implantable intraluminal medical device comprises a structure having interlocking segments formed from at least one polymer, at least one therapeutic agent dispersed throughout the at least one polymer in a concentration of up to thirty percent and at least one radiopaque agent dispersed throughout the at least one polymer in a concentration of up to thirty percent.

In accordance with another aspect, the present invention is directed to an implantable intraluminal medical device. The implantable intraluminal medical device comprises a structure having interlocking segments formed from at least one polymer; and at least one therapeutic agent dispersed throughout the at least one polymer in a concentration of up to thirty percent.

In accordance with another aspect, the present invention is directed to an implantable intraluminal medical device. The implantable intraluminal medical device comprises a structure having interlocking segments formed from at least one polymer, and at least one radiopaque agent dispersed throughout the at least one polymer in a concentration of up to thirty percent.

In accordance with another aspect, the present invention is directed to a method of deploying an intraluminal device. The method comprises the steps of introducing a delivery system in to the vasculature at the treatment site, expanding the intraluminal device utilizing a pressure ranging from about one atmosphere to about ten atmospheres.

In accordance with another aspect, the present invention is directed to a method of sterilizing a drug containing a polymeric intraluminal device. The method comprises the steps placing the polymeric intraluminal device into a sterilization chamber, introducing a sterilization agent into the chamber at a first temperature, a first pressure and a first humidity level for a first period of time, the first temperature not to exceed sixty degrees Celsius and removing the sterilization agent from the chamber by introducing an inert gas into the chamber at a second temperature, a second pressure and a second humidity level for a second period of time.

In accordance with another aspect, the present invention is directed to a method for treating long lesions in the vasculature. The method comprises the steps of positioning a structure having individual segments formed from at least one polymer and comprising at least one therapeutic agent dispersed throughout each of the individual segments in the at least one polymer in a concentration of up to thirty percent; and expanding each of the individual segments to open and support the vasculature.

The biocompatible materials for implantable medical devices of the present invention may be utilized for any number of medical applications, including vessel patency devices, such as vascular stents, biliary stents, ureter stents, vessel occlusion devices such as atrial septal and ventricular septal occluders, patent foramen ovale occluders and orthopedic devices such as fixation devices.

The biocompatible materials of the present invention comprise unique compositions and designed-in properties that enable the fabrication of stents and/or other implantable medical device that are able to withstand a broader range of loading conditions than currently available stents and/or other implantable medical devices. More particularly, the molecular structure designed into the biocompatible materials facilitates the design of stents and/or other implantable medical devices with a wide range of geometries that are adaptable to various loading conditions.

The intraluminal devices of the present invention may be formed out of any number of biocompatible polymeric materials. In order to achieve the desired mechanical properties, the polymeric material, whether in the raw state or in the tubular or sheet state may be physically deformed to achieve a certain degree of alignment of the polymer chains. This alignment may be utilized to enhance the physical and/or mechanical properties of one or more components of the stent.

The intraluminal devices of the present invention may also be formed from blends of polymeric materials, blends of polymeric materials and plasticizers, blends of polymeric materials and therapeutic agents, blends of polymeric materials and radiopaque agents, blends of polymeric materials with both therapeutic and radiopaque agents, blends of polymeric materials with plasticizers and therapeutic agents, blends of polymeric materials with plasticizers and radiopaque agents, blends of polymeric materials with plasticizers, therapeutic agents and radiopaque agents, and/or any combination thereof. By blending materials with different properties, a resultant material may have the beneficial characteristics of each independent material. For example, stiff and brittle materials may be blended with soft and elastomeric materials to create a stiff and tough material. In addition, by blending either or both therapeutic agents and radiopaque agents together with the other materials, higher concentrations of these materials may be achieved as well as a more homogeneous dispersion. Various methods for producing these blends include solvent and melt processing techniques.

### BRIEF DESCRIPTION OF THE DRAWINGS

The foregoing and other features and advantages of the invention will be apparent from the following, more particular description of preferred embodiments of the invention, as illustrated in the accompanying drawings.

FIG. 1 is a planar representation of an exemplary stent fabricated from biocompatible materials in accordance with the present invention.

FIG. 2 is a perspective view of the stent in a closed-configuration in accordance with the present invention;

FIG. 3 is a partial side plan view of the stent of FIG. 1 in the closed configuration in accordance with the present invention;

FIG. 4 is a perspective view of the stent of FIG. 2 in an open configuration in accordance with the present invention;

FIG. 5 is a partial side view of the stent of FIG. 2 in the open configuration in accordance with the present invention;

FIG. 6 is a partial side view of an alternative embodiment of a stent having multiple locking points in a closed configuration in accordance with the present invention;

FIGS. 7A-7E depict partial side views of the stent of FIG. 6 in discrete locked positions during various stages of moving the stent from the closed configuration to an open configuration in accordance with the present invention; and

FIG. 8 is a partial side view of the stent of FIG. 6 in this open configuration in accordance with the present invention.

FIG. 9 is a schematic representation of a stress-strain curve of a stiff and brittle material and a plasticized material in accordance with the present invention.

FIG. 10 is a schematic representation of a stress-strain curve of a stiff and brittle material, a soft and elastomeric material and a blend of the stiff and elastomeric material in accordance with the present invention.

### DETAILED DESCRIPTION OF THE PREFERRED EMBODIMENTS

Implantable medical devices may be fabricated from any number of suitable biocompatible materials, including polymeric materials. The internal structure of these polymeric materials may be altered utilizing mechanical and/or chemical manipulation of the polymers. These internal structure modifications may be utilized to create devices having specific gross characteristics such as crystalline and amorphous morphology and orientation as is explained in detail subsequently. Although the present invention applies to any number of implantable medical devices, for ease of explanation, the following detailed description will focus on an exemplary stent.

In accordance with the present invention, implantable medical devices may be fabricated from any number of biocompatible materials, including polymeric materials. These polymeric materials may be non-degradable, biodegradable and/or bioabsorbable. These polymeric materials may be formed from single polymers, blends of polymers and blends of polymers and plasticizers. In addition, other agents such as drugs and/or radiopaque agents may be blended with the materials described above or affixed or otherwise added thereto. A number of chemical and/or physical processes may be utilized to alter the chemical and physical properties of the materials and ultimately the final devices.

### Exemplary Devices

A stent is commonly used as a tubular structure left inside the lumen of a duct to relieve an obstruction. Commonly, stents are inserted into the lumen in a non-expanded form and are then expanded autonomously (or with the aid of a second device) in situ. When used in coronary artery procedures for relieving stenosis, stents are placed percutaneously through the femoral artery. In this type of procedure, stents are delivered on a catheter and are either self-expanding or, in the majority of cases, expanded by a balloon. Self-expanding stents do not need a balloon to be deployed. Rather the stents are constructed using metals with spring-like or superelastic properties (i.e., Nitinol), which inherently exhibit constant radial support. Self-expanding stents are also often used in vessels close to the skin (i.e., carotid arteries) or vessels that can experience a lot of movement (i.e., popliteal artery). Due to a natural elastic recoil, self-expanding stents withstand pressure or shifting and maintain their shape. The self-expanding stent is typically introduced via a catheter wherein an outer sheath is retracted once the stent is in the proper location to allow the stent to expand into position.

As mentioned above, the typical method of expansion for balloon expanded stents occurs through the use of a catheter mounted angioplasty balloon, which is inflated within the stenosed vessel or body passageway, in order to shear and disrupt the obstructions associated with the wall components of the vessel and to obtain an enlarged lumen.

Balloon-expandable stents of different sizes involve crimping the device onto an angioplasty balloon with different crimp profiles. The stent takes shape as the balloon is inflated and remains in place when the balloon and delivery system are deflated and removed. Inflation pressures for the balloon range from about 6 atmospheres to about 20 atmospheres for metallic stents and from about 1 atmosphere to about 20 atmospheres for polymeric stents. In a preferred embodiment, the inflation pressure may be in the range from about one atmosphere to about ten atmospheres for complete expansion. The stent and the balloon are introduced via a catheter.

In addition, balloon-expandable stents are available either pre-mounted or unmounted. A pre-mounted system has the stent already crimped on a balloon, while an unmounted system gives the physician the option as to what combination of devices (catheters and stents) to use. Accordingly, for these types of procedures, the stent is first introduced into the blood vessel on a balloon catheter. Then, the balloon is inflated causing the stent to expand and press against the vessel wall. After expanding the stent, the balloon is deflated and withdrawn from the vessel together with the catheter. Once the balloon is withdrawn, the stent stays in place permanently, holding the vessel open and improving the flow of blood.

In the absence of a stent, restenosis may occur as a result of elastic recoil of the stenotic lesion. Although a number of stent designs have been reported, these designs have suffered from a number of limitations. Some of these limitations include design limitations resulting in low radial strength, decrease in the length of the stent upon deployment, i.e. foreshortening, and high degree of axial compression experienced by the stent.

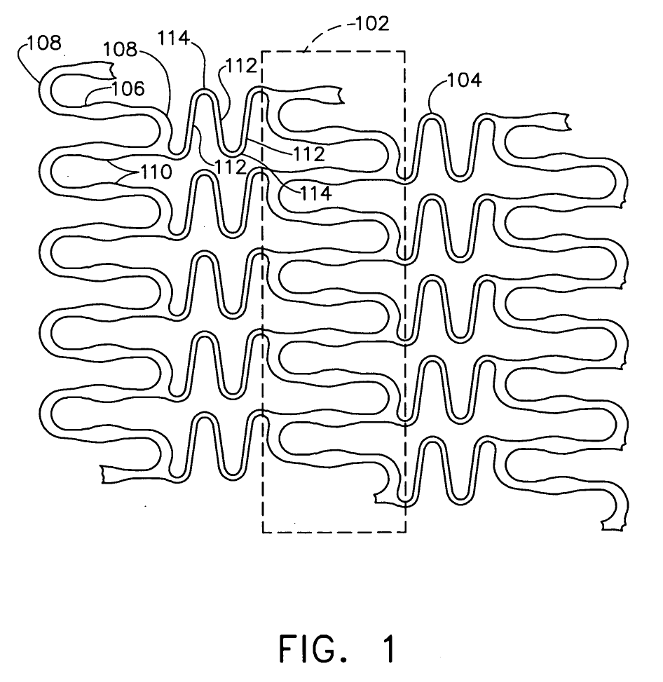

Referring to FIG. 1, there is illustrated a partial planar view of an exemplary stent 100 in accordance with the present invention. The exemplary stent 100 comprises a plurality of hoop components 102 interconnected by a plurality of flexible connectors 104. The hoop components 102 are formed as a continuous series of substantially longitudinally or axially oriented radial strut members 106 and alternating substantially circumferentially oriented radial arc members 108. Although shown in planar view, the hoop components 102 are essentially ring members that are linked together by the flexible connectors 104 to form a substantially tubular stent structure. The combination of radial strut members 106 and alternating radial arc members 108 form a substantially sinusoidal pattern. Although the hoop components 102 may be designed with any number of design features and assume any number of configurations, in the exemplary embodiment, the radial strut members 106 are wider in their central regions 110. This design feature may be utilized for a number of purposes, including, increased surface area for drug delivery.

The flexible connectors 104 are formed from a continuous series of flexible strut members 112 and alternating flexible arc members 114. The flexible connectors 104, as described above, connect adjacent hoop components 102 together. In this exemplary embodiment, the flexible connectors 104 have a substantially N-shape with one end being connected to a radial arc member on one hoop component and the other end being connected to a radial arc member on an adjacent hoop component. As with the hoop components 102, the flexible connectors 104 may comprise any number of design features and any number of configurations. In the exemplary embodiment, the ends of the flexible connectors 104 are connected to different portions of the radial arc members of adjacent hoop components for ease of nesting during crimping of the stent. It is interesting to note that with this exemplary configuration, the radial arcs on adjacent hoop components are slightly out of phase, while the radial arcs on every other hoop component are substantially in phase. In addition, it is important to note that not every radial arc on each hoop component need be connected to every radial arc on the adjacent hoop component.

It is important to note that any number of designs may be utilized for the flexible connectors or connectors in an intraluminal scaffold or stent. For example, in the design described above, the connector comprises two elements, substantially longitudinally oriented strut members and flexible arc members. In alternate designs, however, the connectors may comprise only a substantially longitudinally oriented strut member and no flexible arc member or a flexible arc connector and no substantially longitudinally oriented strut member.

The substantially tubular structure of the stent 100 provides either temporary or permanent scaffolding for maintaining patency of substantially tubular organs, such as arteries. The stent 100 comprises a luminal surface and an abluminal surface. The distance between the two surfaces defines the wall thickness. The stent 100 has an unexpanded diameter for delivery and an expanded diameter, which roughly corresponds to the normal diameter of the organ into which it is delivered. As tubular organs such as arteries may vary in diameter, different size stents having different sets of unexpanded and expanded diameters may be designed without departing from the spirit of the present invention. As described herein, the stent 100 may be formed from any number of polymeric materials. These stents may be prepared from other materials such as polymer-metal composites. Exemplary materials include the utilization of biostable metal-biostable polymers, biostable metal-bioabsorbable polymers, bioabsorbable metal-biostable polymers, and bioabsorbable metal-bioabsorbable polymers. These materials may be used for the full stent or portions thereof.

In accordance with another embodiment, a stent having interlocking elements with multiple locking points may be utilized for stenting a vessel.

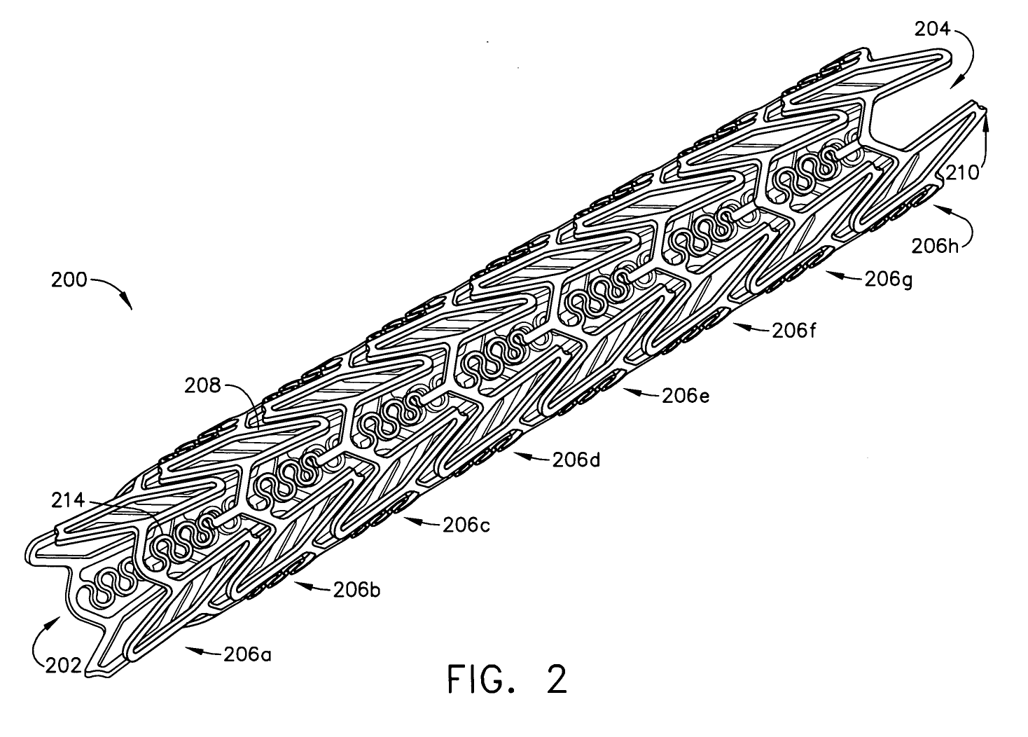

In FIGS. 2-5, a stent 206 that is an expandable prosthesis for a body passageway is illustrated. It should be understood that the terms “stent” and “prosthesis” are interchangeably used to some extent in describing the present invention, insofar as the method, apparatus, and structures of the present invention may be utilized not only in connection with an expandable intraluminal vascular graft for expanding partially occluded segments of a blood vessel, duct or body passageways, such as within an organ, but may so be utilized for many other purposes as an expandable prosthesis for many other types of body passageways. For example, expandable prostheses may also be used for such purposes as: (1) supportive graft placement within blocked arteries opened by transluminal recanalization, but which are likely to collapse in the absence of internal support; (2) similar use following catheter passage through mediastinal and other veins occluded by inoperable cancers; (3) reinforcement of catheter created intrahepatic communications between portal and hepatic veins in patients suffering from portal hypertension; (4) supportive graft placement of narrowing of the esophagus, the intestine, the ureters, the urethra, etc.; (5) intraluminally bypassing a defect such as an aneurysm or blockage within a vessel or organ; and (6) supportive graft reinforcement of reopened and previously obstructed bile ducts. Accordingly, use of the term “prosthesis” encompasses the foregoing usages within various types of body passageways, and the use of the term “intraluminal graft” encompasses use for expanding the lumen of a body passageway. Further in this regard, the term “body passageway” encompasses any lumen or duct within the human body, such as those previously described, as well as any vein, artery, or blood vessel within the human vascular system.

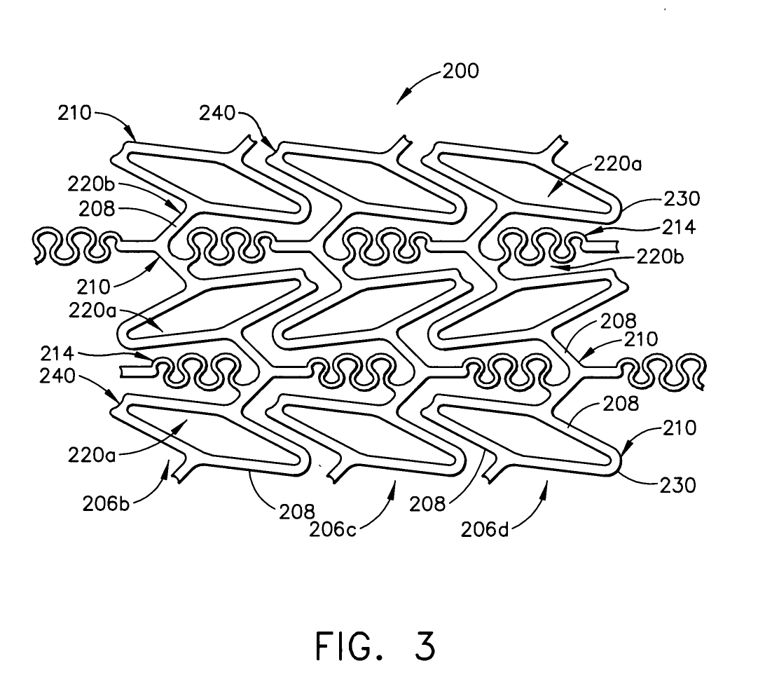

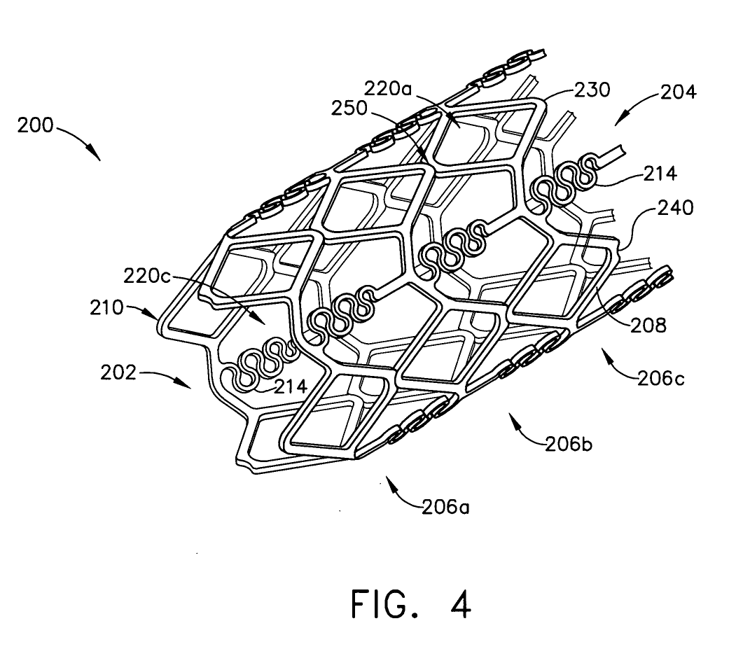

The stent 200 is an expandable lattice structure made of any suitable material, which is compatible with the human body and the bodily fluids (not shown) with which the stent 200 may come into contact. The lattice structure is an arrangement of interconnecting elements made of a material which has the requisite strength and elasticity characteristics to permit the tubular shaped stent 200 to be moved or expanded from a closed (crimped) position or configuration shown in FIGS. 2 and 3 to an expanded or open position or configuration shown in FIGS. 3 and 4. Some examples of materials that are used for the fabrication of the stent 300 include silver, tantalum, stainless steel, gold, titanium or any type of plastic material having the requisite characteristics previously described. Based on the interlocking design of the stent 200 (greater detail provided later in this disclosure), when the stent 200 is deployed or expanded to its open position, even materials that tend to recoil to a smaller diameter or exhibit crushing or deformation-like properties are used for the stent 200 in accordance with the present invention. These are materials that are not used in traditional (prior art) stent designs. Some examples of these non-traditional stent materials that are used for the stent 200 in accordance with the present invention include deformable plastics, plastics that exhibit crushing or recoil upon deployment of the stent or polymers such as biodegradable polymers. Thus, the stent 200 in accordance with the present invention is also made of these type of plastics or polymers to include biodegradable polymers. Additionally, the biodegradable polymers used as material for the stent 200 can be drug eluting polymers capable of eluting a therapeutic and/or pharmaceutical agent according to any desired release profile.

In one embodiment, the stent is fabricated from 316L stainless steel alloy. In a preferred embodiment, the stent 200 comprises a superelastic alloy such as nickel titanium (NiTi, e.g., Nitinol). More preferably, the stent 200 is formed from an alloy comprising from about 50.5 to 60.0 percent Ni by atomic weight and the remainder Ti. Even more preferably, the stent 200 is formed from an alloy comprising about 55 percent Ni and about 45 percent Ti. The stent 200 is preferably designed such that it is superelastic at body temperature, and preferably has an Af temperature in the range from about 24 degrees C. to about 37 degrees C. The superelastic design of the stent 200 makes it crush recoverable and thus suitable as a stent or frame for any number of vascular devices for different applications.

The stent 200 comprises a tubular configuration formed by a lattice of interconnecting elements defining a substantially cylindrical configuration and having front and back open ends 202, 204 and defining a longitudinal axis extending therebetween. In its closed configuration, the stent 200 has a first diameter for insertion into a patient and navigation through the vessels and, in its open configuration, a second diameter, as shown in FIG. 4, for deployment into the target area of a vessel with the second diameter being greater than the first diameter. The stent 200 comprises a plurality of adjacent hoops 206a-206h extending between the front and back ends 202, 204. The stent 200 comprises any combination or number of hoops 206. The hoops 206a-206h include a plurality of longitudinally arranged struts 208 and a plurality of loops 210 connecting adjacent struts 208. Adjacent struts 208 or loops 210 are connected at opposite ends by flexible links 214 which can be any pattern such as sinusoidal shape, straight (linear) shape or a substantially S-shaped or Z-shaped pattern. The plurality of loops 210 have a substantially curved configuration.

The flexible links 214 serve as bridges, which connect adjacent hoops 206a-206h at the struts 208 or loops 210. Each flexible link comprises two ends wherein one end of each link 214 is attached to one strut 208 or one loop 210 on one hoop 206a and the other end of the link 214 is attached to one strut 208 or one loop 210 on an adjacent hoop 206b, etc.

The above-described geometry better distributes strain throughout the stent 200, prevents metal to metal contact where the stent 200 is bent, and minimizes the opening between the features of the stent 200; namely, struts 208, loops 210 and flexible links 214. The number of and nature of the design of the struts, loops and flexible links are important design factors when determining the working properties and fatigue life properties of the stent 200. It was previously thought that in order to improve the rigidity of the stent, struts should be large, and thus there should be fewer struts 208 per hoop 206a-206h. However, it is now known that stents 200 having smaller struts 208 and more struts 208 per hoop 206a-206h improve the construction of the stent 200 and provide greater rigidity. Preferably, each hoop 206a-206h has between twenty-four (24) to thirty-six (36) or more struts 208. It has been determined that a stent having a ratio of number of struts per hoop to strut length which is greater than four hundred has increased rigidity over prior art stents which typically have a ratio of under two hundred. The length of a strut is measured in its compressed state (closed configuration) parallel to the longitudinal axis of the stent 200 as illustrated in FIG. 2.

FIG. 4 illustrates the stent 200 in its open or expanded state. As may be seen from a comparison between the stent 200 configuration illustrated in FIG. 2 and the stent 200 configuration illustrated in FIG. 4, the geometry of the stent 200 changes quite significantly as it is deployed from its unexpanded state (closed or crimped configuration/position) to its expanded state (open or expanded configuration/position). As the stent 200 undergoes diametric change, the strut angle and strain levels in the loops 210 and flexible links 214 are affected. Preferably, all of the stent features will strain in a predictable manner so that the stent 200 is reliable and uniform in strength. In addition, it is preferable to minimize the maximum strain experienced by the struts 208, loops 210 and flexible links 214 since Nitinol properties are more generally limited by strain rather than by stress. The embodiment illustrated in FIGS. 2-5 has a design to help minimize forces such as strain.

As best illustrated in FIG. 3, the stent 200, in the closed-configuration (crimped configuration wherein the stent 200 is crimped on the stent delivery device such as a catheter), has a plurality of pre-configured cells 220a. Each pre-configured cell 220a is defined by the struts 208 and loops 210 connected to each other respectively thereby defining an open area in the stent lattice 200. This open area is a space identified as the pre-configured cell 220a.

Each hoop 206a-206h has one or more (or a plurality of) pre-configured cells 220a. In one embodiment according to the present invention, the pre-configured cell 220a is a diamond-shaped area or space. However, it is contemplated in accordance with the present invention that the pre-configured cell 220a take the form of any desired alternative shape.

Additionally, the stent lattice 200 also includes at least one (or a plurality of) partial cells 220b. Each partial cell 220b is defined by struts 208 and one loop 210 of the respective hoops 206a-206h. In one embodiment according to the present invention, the partial cell 220b defines a semi-enclosed area or space having an open end in direct communication with a loop 210 from an adjacent hoop 206a-206h. In this embodiment according to the present invention, the flexible link 214 connects adjacent hoops, for example hoop 206b to hoop 206c, by having one end of flexible link 214 connected to an inner surface of loop 210 of a partial cell 220b of the hoop 206b and the opposite end of the flexible link 214 connected to loop 210 of the adjacent hoop 206c. Thus, in this embodiment, the flexible link 214 extends from one end of the partial cell 220b, for instance, of hoop 206b and extends through the semi-enclosed area of the partial cell 220b and is connected to loop 210 of the adjacent hoop 206c. In this embodiment according to the present invention, the flexible links 214 are connected between adjacent hoops 206a-206h by extension through the partial cells 220b. Additionally, the partial cell 220b is not only a semi-enclosed area or space defined by struts 208 and one loop 210 of each hoop 206, but the partial cell 220b may take the form of any desired semi-enclosed shape.

In this embodiment according to the present invention, each partial cell 220b of the stent lattice 200 exists while the stent 200 is in its crimped state or closed configuration, i.e. crimped to the delivery device such as a catheter.

Moreover, in one embodiment according to the present invention, each pre-configured cell 220a has one loop 210 terminating in a male end 230 and the other loop defining the pre-configured cell 220a terminating in a female and 240. Thus, in this embodiment in accordance with the present invention, the male end 230 of one loop 210 and the female end 240 of the other loop 210 of the pre-configured cell 220a are positioned opposite each other thereby defining opposite ends of the pre-configured cell 220a, for example opposite ends of the diamond-shaped area in this embodiment.

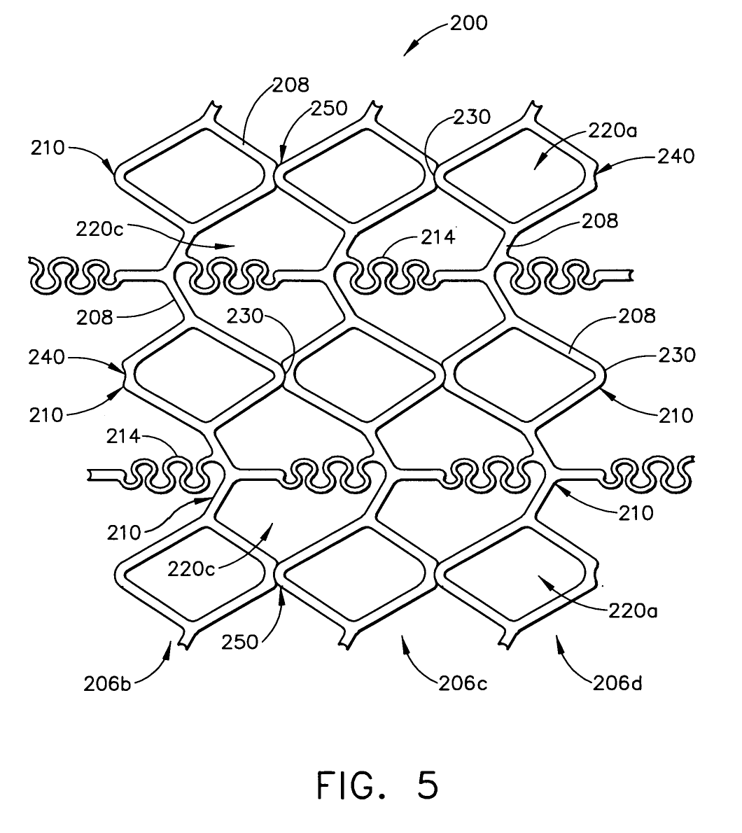

In one embodiment in accordance with the present invention, the male end 230 has a substantially convex configuration and the female end 240 has a substantially concave configuration. In general, the female end 240 is designed such that it is shaped to receive and mateably connect with the male end 230. Accordingly, in this embodiment, the substantially concave surface of the female end 240 mateably connects with the substantially convex shape of the male end 230 when the stent lattice 100 is moved to the open configuration or state (deployed or expanded state) such as shown in FIGS. 4 and 5.

As best illustrated in FIG. 5, when the stent lattice 200 is deployed or expanded to its open position or configuration, the male end 230 of the loop 210 of one hoop 206, for example 206b, mateably connects with the female end 240 of an opposite loop 210 of an adjacent hoop, for example 206c, thereby forming a locked joint 250. The male end 230 and the female end 240 may take the form of any desired shape or configuration that permits the male end 230 to mateably connect with the female end 140 in order to form the locked joint 250. For example, the male end 230 and the female end 240 may be shaped respectively in order to form portions of a dove-tail such that the locked joint 250 has or forms a dove-tail configuration. Other shapes for the male end 230 and female end 240 forming the locked joint 250 are also contemplated herein.

Accordingly, when the stent lattice 200 is deployed or expanded to the open position (open configuration of the stent 200), adjacent hoops 206a-206h interlock with each other at the newly formed joints 250 mateably connecting adjacent hoops 206a-206h. For example, when the stent lattice 200 is moved to its open configuration, the hoop 206b mateably connects or interlocks with the adjacent hoop 206c and the hoop 206c interlocks with the adjacent hoop 206d, etc. Thus, the points of interlocking or mateable connection are located at the newly formed locked joint 250 between each pair of adjacent hoops 206 as shown. Thus, each locked joint 250 is formed by at least one loop 210 of one hoop 206 (for example 206b, wherein the male end 230 of this loop 210 mateably connects with the female end 240 of another loop 210), i.e. an adjacent loop on an adjacent hoop 206, for example loop 210 on the hoop 206c which is directly opposed from the male end 230 of loop 210 of the hoop 206b. Therefore, the adjacent hoops 206a-206h, are mateably connected to or locked to each other respectively at each locked joint 250 formed in a manner such as described above.

Upon the mateable connection or linking of the male end 230 to the female end 240 (on the loops 210 of adjacent hoops 206), a formed cell 220c is created or formed between adjacent locked joints 250 form by a pair of interlocking, adjacent hoops 206, for example, 206a and 206b, etc. Each formed cell 220c is a fully enclosed area or space defined by the struts 208 loops 210 and locked joints 250 formed by the adjacent hoops 206, i.e. linking of hoop 206a to hoop 106b, linking of hoop 206b to adjacent hoop 206c, etc. Accordingly, the partial cell 220b (FIG. 2) of the stent lattice 200 in its crimped configuration, becomes the formed cell 220c when linked or coupled by the locked joint 250 between adjacent hoops 206 as shown in FIG. 4.

In accordance with the present invention, the stent 200 has flexible links 210 that may be on one or more of the following components of the stent lattice: the hoops 206a-206h, the loops 210, and/or the struts 208. Moreover, the components of the stent lattice, i.e. hoops, loops, struts and flexible links, have drug coatings or drug and polymer coating combinations that are used to deliver drugs, i.e. therapeutic and/or pharmaceutical agents including: antiproliferative/antimitotic agents including natural products such as vinca alkaloids (i.e. vinblastine, vincristine, and vinorelbine), paclitaxel, epidipodophyllotoxins (i.e. etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents such as G(GP)II_{b}IIIₐ inhibitors and vitronectin receptor antagonists; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nirtosoureas (carmustine (BCNU) and analogs, streptozocin), trazenes—dacarbazinine (DTIC); antiproliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine {cladribine}); platinum coordination complexes (cisplatin, carboplatin), procarbazine, hydroxyurea, mitotane, aminoglutethimide; hormones (i.e. estrogen); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); antiinflammatory: such as adrenocortical steroids (cortisol, cortisone, fludrocortisone, prednisone, prednisolone, 6α-methylprednisolone, triamcinolone, betamethasone, and dexamethasone), non-steroidal agents (salicylic acid derivatives i.e. aspirin; para-aminophenol derivatives i.e. acetominophen; indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives: (cyclosporine, tacrolimus (FK-506), sirolimus (rapamycin), azathioprine, mycophenolate mofetil); angiogenic agents: vascular endothelial growth factor (VEGF), fibroblast growth factor (FGF) platelet derived growth factor (PDGF), erythropoetin; angiotensin-receptor blocker; nitric oxide donors; anti-sense oligionucleotides and combinations thereof; cell cycle inhibitors, mTOR inhibitors, and growth factor signal transduction kinase inhibitors. It is important to note that one or more of the lattice components (e.g. hoops, loops, struts and flexible links) are coated with one or more of the drug coatings or drug and polymer coating combinations. Additionally, as mentioned above, the stent 100 is alternatively made of a polymer material itself such as a biodegradable material capable of containing and eluting one or more drugs, in any combination, in accordance with a specific or desired drug release profile.

The method of utilizing the stent 200 according to the present invention includes first identifying a location, for example, a site within the vessel in a patient's body for deployment of the stent 200. Upon identifying the desired deployment location, for example a stenotic lesion or vulnerable plaque site, a delivery device, such as a catheter carrying the stent 200 crimped to a distal end of the catheter such that the stent 200 is in its closed configuration, is inserted within the vessel in the patient's body. The catheter is used to traverse the vessel until reaching the desired location (site) wherein the distal end of the catheter is positioned at the desired location (site), for instance the lesion, within the vessel. At this point, the stent 200 is deployed to its open configuration by expanding the stent 200 such as by inflation if the stent 200 is a balloon expandable stent or by uncovering or release of the stent 200 if the stent 200 is a self-expanding (crush recoverable) type stent. If a cover is utilized to further protect and secure the stent 200 to the catheter distal end when the stent 100 is a self-expanding stent, the cover is removed from the distal end of the catheter prior to expansion of the stent 200, for instance, through use of an expandable member such as an inflatable balloon.

Upon expanding the stent 200 to its open configuration, the expandable member (balloon) is then collapsed, for instance through deflation of the expandable member, whereby the catheter is removed from the deployment site of the vessel and patient's body altogether.

As mentioned previously, the unique design of the stent 200, i.e. the interlocking of adjacent hoops 106 upon deployment of the stent 200, allows for a wide array of materials, not previously used with prior art stents, to be used with the stent 200 in accordance with the present invention. These include materials normally prone to crushing, deformation or recoil upon deployment of the stent. These materials include plastics and polymers to include biodegradable polymers such as drug eluting polymers.

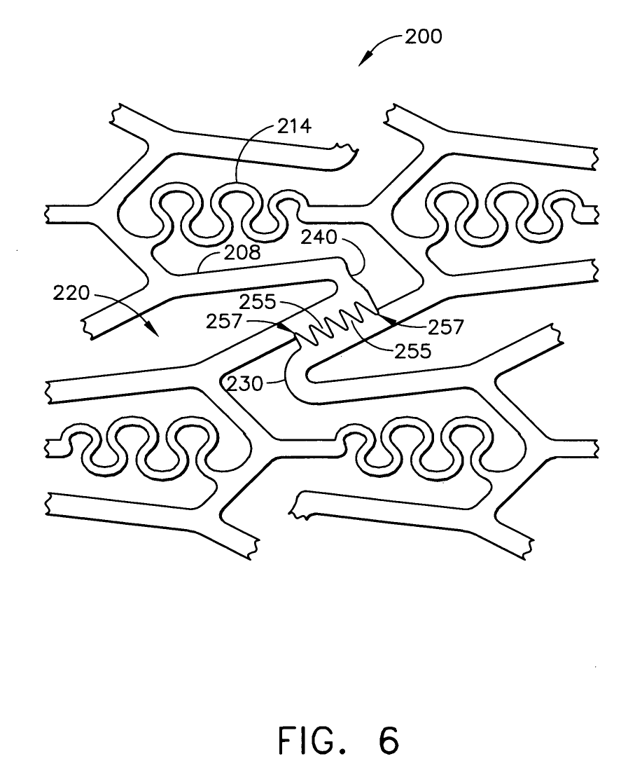

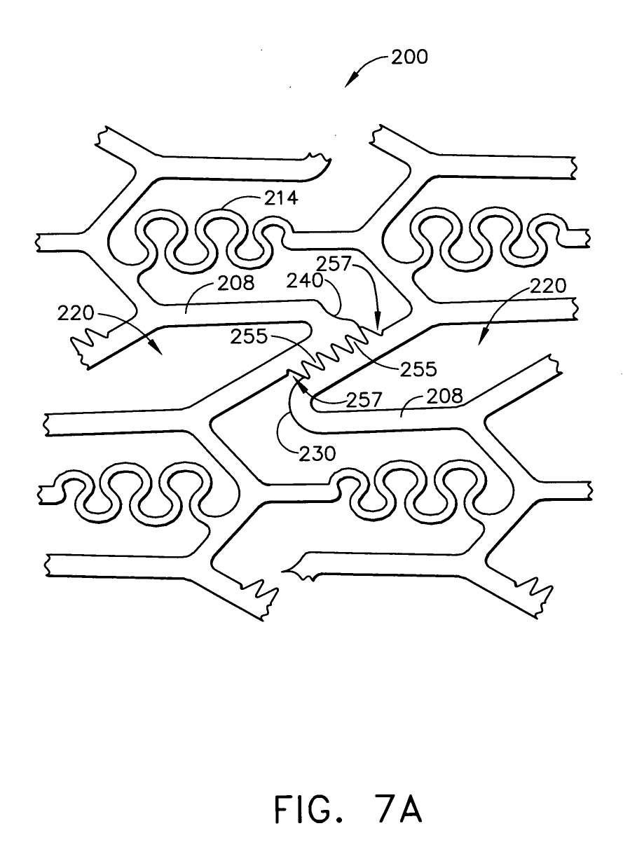

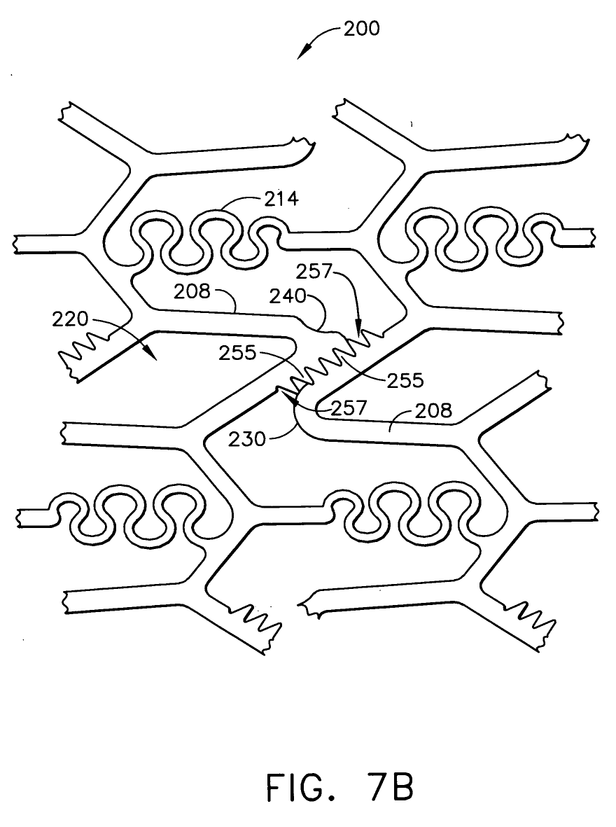

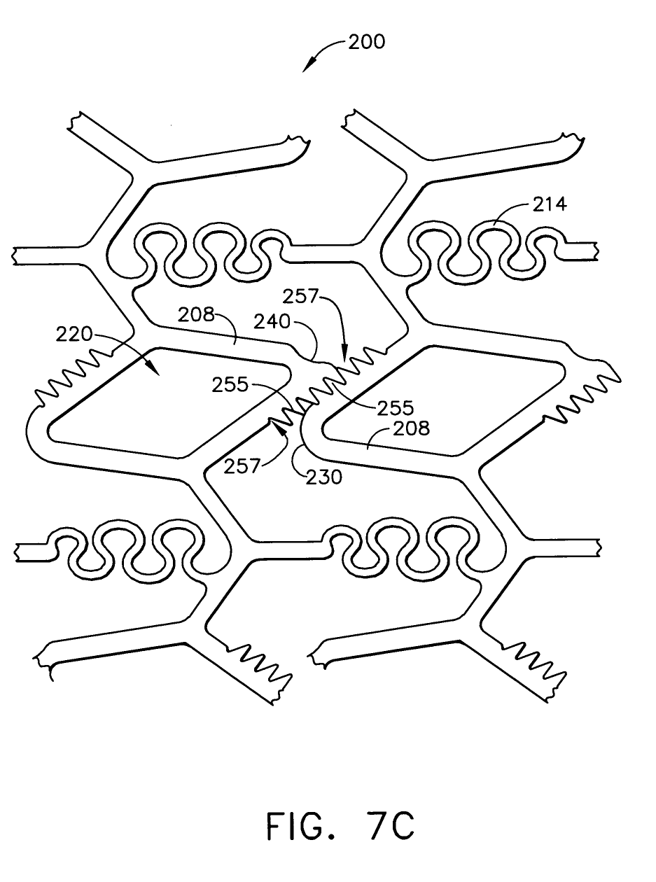

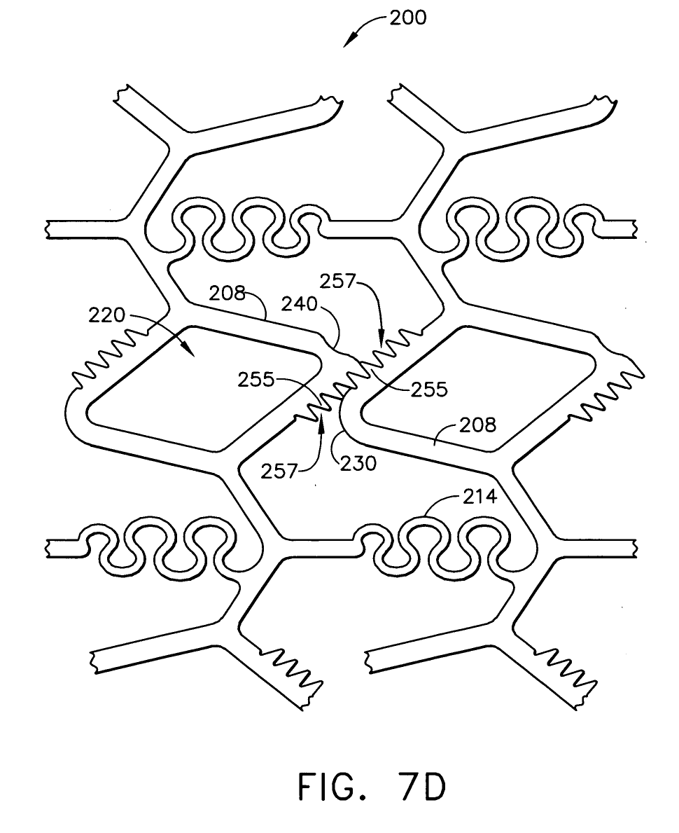

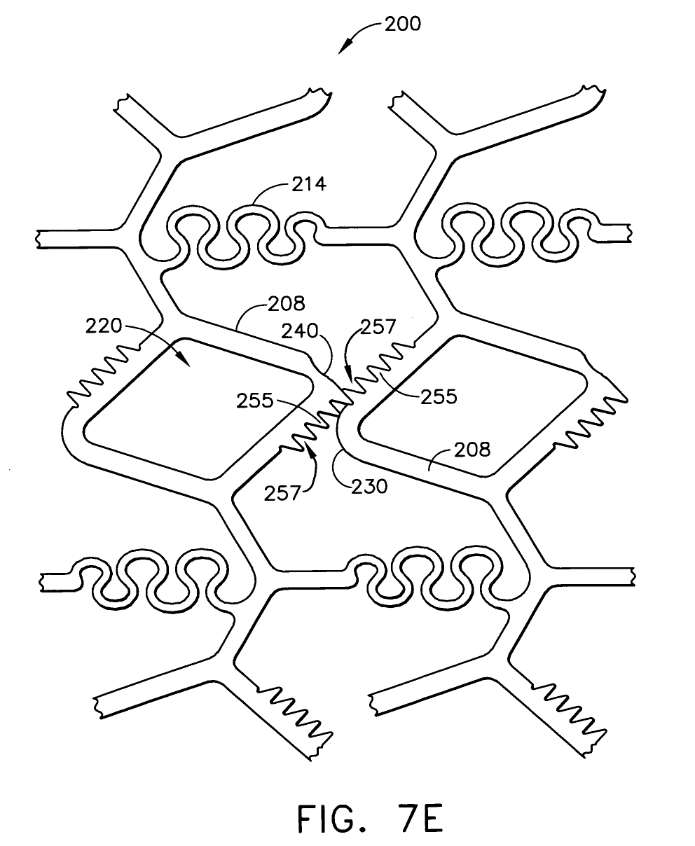

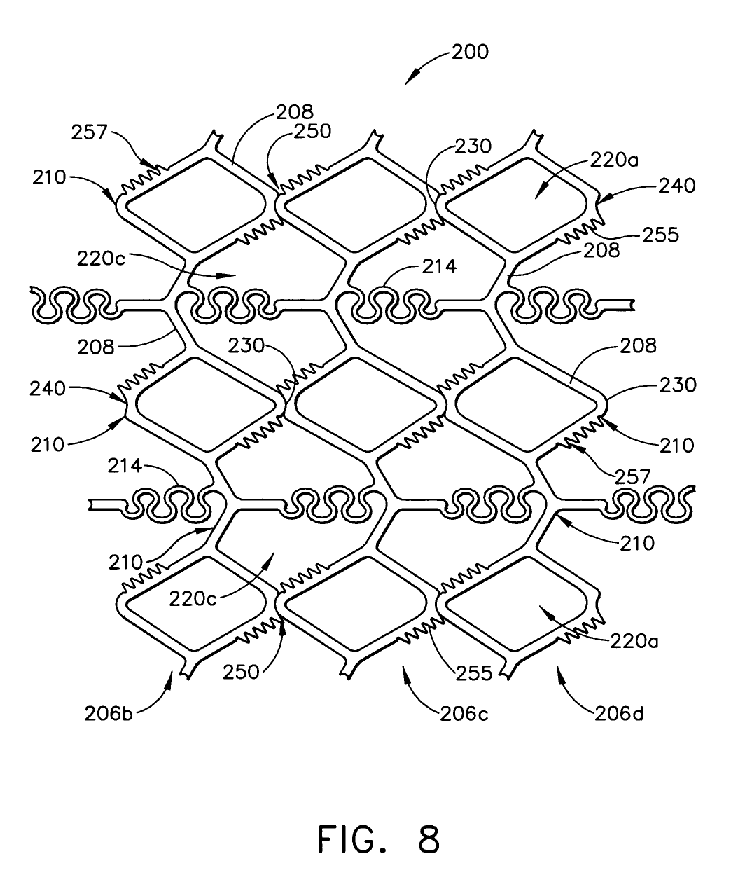

An alternative embodiment for the stent 200 in accordance with the present invention is best depicted in FIG. 6, FIGS. 7A-7E, and FIG. 8. The stent 200 in accordance with this embodiment of the present invention has the same or substantially similar features, elements and their functions as detailed above for the stent embodiment of FIGS. 2-5 above. Likewise, the same reference numerals are used to designate like or similar features and their function for the stent embodiment of FIGS. 6, 7A-7E and 8 in accordance with the present invention.

FIG. 6 and FIG. 8 are partial, enlarged views that illustrate the stent 200 having one or more struts 208 on adjacent hoops 206 wherein these struts 208 have a plurality of teeth 255 arranged along the outer edge or outer surface of these struts 208. The teeth 255 of adjacent struts 208 of adjacent hoops 206 as shown are designed such that the teeth 255 of the respective struts 208 are in interlocking engagement (mateably connectable) or mesh with each other at a plurality of locking points 257. The locking points 257 are defined by a tip of one of the teeth 255 received in a tip receiving area or notch on the opposite strut 208 (of an adjacent hoop 206) wherein the area of this strut 208 is shaped to receive the tips of the teeth 255 of the opposite strut 208 of the adjacent hoop 206.

Accordingly, this arrangement as shown in FIG. 6, clearly depicts interlocking adjacent struts 208. All teeth 255 of one strut 208 are moveably received in the tip receiving areas 257, i.e. locking points, when the stent 200 is in the closed configuration. Accordingly, when the stent 200 is in the closed configuration, all teeth 255 of one strut 208 are seated or fit within their locking points 257 of the opposed strut 208 on the adjacent hoop 206.

Although FIG. 6, FIG. 7A-7E, and FIG. 8 depict the interlocking adjacent struts 208 having a total of five teeth respectively, the adjacent interlocking struts 208 are not limited to any particular number of teeth, but rather comprise one or more teeth respectively as desired. Moreover, the present invention is not limited to the saw tooth or serrated edge embodiment 255 for the interlocking adjacent struts 208, but rather, includes any configuration (for example, sinusoidal, dove tail, tongue and groove, etc.) for the interlocking teeth 255, so long as the interlocking adjacent struts 208 have multiple and discrete locking points 257 that permit the stent 200 to be opened to a plurality of discrete or separate locked positions. Each of these discrete or separate locked positions serve as the open configuration for stent 200 (FIGS. 5-8) if desired.

FIGS. 7A-7E depict the stent 200 at various stages of locked movement as the stent 200 is lockably moved from its closed configuration (FIG. 6) to its final open configuration (FIG. 8). As shown in FIGS. 7A-7E, as the stent 200 is expanded from its closed configuration to its open configuration, each notch 257 is exposed as the teeth 255 of the adjacent interlocking struts 208 are interlockingly moved, indexed or ratcheted through the various locking points 257 as shown. It is important to note that stent 200 can be either locked in its closed configuration as shown in FIG. 6 or unlocked in its closed configuration; i.e. no teeth 255 engaged with a respective notch 257 (not shown).

The interlocking adjacent struts 208 each have an interlockable edge, i.e. a serrated edge or teeth 255, in this example, along their common sides that allow for multiple locking interactions between the diamond-shape cells 120 and 120a (FIG. 7) as the diameter of the stent 200 is increased, e.g. through expanding the stent 100 from its closed configuration (FIG. 6) to its final open configuration (FIG. 8). As shown in FIGS. 7A-7E, the teeth or serrated edges 255 engage the opposed struts 208 of adjacent hoops 206 at their common interlockable edges such that the two adjacent cells 220 on the respective adjacent hoops 206 move parallel to one another during expansion of the stent 200.

In accordance with the present invention, the stent embodiment depicted in FIG. 6, FIGS. 7A-7E and FIG. 8 results in a stent having a highly selectable, customizable locking design that permits the stent 200 to be opened to any desired locked diameter, i.e. an open position that is a locked position at various diameter sizes.

Accordingly, as illustrated, the stent 200 is deployed to a plurality of distinct, variable diameters (increasing size diameters). For example, the stent 200 in accordance with the present invention is lockingly expandable from its closed configuration (FIG. 6) to one of six different and distinct stent diameters (open configuration) as shown in FIGS. 7A, 7B, 7D, 7E, and FIG. 8 respectively. As mentioned above, these different and distinct stent diameters increase in size at each different locking point 257 and 250 (FIG. 8) respectively.

Moreover, similar to the stent 200 depicted in FIGS. 2-5, the alternative embodiment of the stent 200 depicted in FIGS. 6, 7A-7E and 8, is also made of these previously described material to include alloys such as stainless steel and nickel titanium (NiTi) or polymers such as biodegradable polymers. Additionally, the stent 100 embodiment depicted in FIGS. 6, 7A-7E and FIG. 8, also comprise a drug or therapeutic agent such as those described previously in this disclosure which include rapamycin, paclitaxel or any of the other previously identified therapeutic agents chemical compounds, biological molecules, nucleic acids such as DNA and RNA, peptides, proteins or combinations thereof.

The stent 200 can be made from biodegradable or bioabsorbable polymer compositions. The type of polymers used can degrade via different mechanisms such as bulk or surface erosion. Bulk erodible polymers include aliphatic polyesters such poly(lactic acid); poly(glycolic acid); poly(caprolactone); poly(p-dioxanone) and poly(trimethylene carbonate); and their copolymers and blends. Other polymers can include amino acid derived polymers; phosphorous containing polymers [e.g., poly(phosphoesters)] and poly(ester amide). Surface erodible polymers include polyanhydrides and polyorthoesters. The stent 200 can be made from combinations of bulk and surface erodible polymers to control the degradation mechanism of the stent. For example, the regions (e.g., interlocks 255 and 257) that are under high stress can be made from a polymer that will retain strength for longer periods of time, as these will degrade earlier than other regions with low stress. The selection of the polymers will determine the absorption of stents 200 that can be very short (few weeks) and long (weeks to months).

The bioabsorbable compositions to prepare the stent 200 will also include drug and radiopaque materials. The amount of drug can range from about 1 to 30 percent as an example, although the amount of drug loading can comprise any desired percentage. The stent 200 will carry more drug than a polymer-coated stent. The drug will release by diffusion and during degradation of the stent 200. The amount of drug release will be for a longer period of time to treat local and diffuse lesions; and for regional delivery for arterial branches to treat diseases such as vulnerable plaque. Radiopaque additives can include barium sulfate and bismuth subcarbonate and the amount can be from 5 to 30 percent as an example.

Other radiopaque materials include gold particles and iodine compounds. The particle size of these radiopaque materials can vary from nanometers to microns. The benefits of small particle size is to avoid any reduction in the mechanical properties and to improve the toughness values of the devices. Upon polymer absorption, small particles will also not have any adverse effects on surrounding tissues.

The tubes to prepare bioabsorbable stents 200 can be fabricated either by melt or solvent processing. The preferred method will be solvent processing, especially for the stents that will contain drug. These tubes can be converted to the desired design by excimer laser processing. Other methods to fabricate the stent can be injection molding using supercritical fluids such as carbon dioxide.

The bibabsorbable stents can be delivered by balloon expansion; self-expansion; or a balloon assist self expansion delivery system. The benefit of using the combination system is that the stent does not have to be crimped to lower profiles and upon deployment the stent will self expand to a certain value and can be further expanded to the desired dimension by balloon expansion in accordance with the present invention as best shown in FIGS. 5-8.

In accordance with the present invention, the embodiment of the stent 200 depicted in FIG. 6, FIGS. 7A-7E and FIG. 8 also provide for increased radial strength for the stent 200 such that the mechanical locking action of the cells 220 and 220a increase the radial strength of the stent 200 in a manner that exceeds the radial strength associated with the prior art stent designs.

Moreover, since the substantially diamond-shaped cells 220 and 220a of the stent 200 in accordance with the present invention are not connected to one another along the axis of the stent, the length of the stent 200 will not decrease or will only exhibit minimal foreshortening as these cells 220 and 220a contract upon deployment of the stent 200.

Furthermore, the mechanical locking action of the cells 220 and 220a prevent the stent 200 from compressing axially, i.e. compression along the longitudinal axis of the stent 200 thereby resulting in increased column strength for the stent 200 in a manner that exceeds the column strength normally associated with the prior art stent designs.

Furthermore, the interlocking adjacent struts 208, due to their respective serrated edges 255 and locking points 257 assist in locking the stent 200 at its smallest diameter while the stent 200 is crimped onto a delivery device such as a catheter, i.e. while the stent 200 is crimped onto the balloon of the delivery catheter. Accordingly, this mating or interlocking of the interlocking adjacent struts 208 (due to their serrated edges 255) prevents the stent 200 from expanding or deploying prematurely until the moment where sufficient force is applied by the inflation of the balloon in order to overcome the resistance caused by the interlocking of the serrations of teeth 255 of the interlocking adjacent struts 208.

Additionally, in accordance with the present invention, the interlocking adjacent struts 208 can have teeth 255 of any desired shape or configuration and any desired number of serrations along the common side of each diamond-shaped cell 220 and 220a in order to increase or decrease the amount of force that is required to either initiate expansion of the stent 200 or to customize or tailor the radial strength of the stent 200 at each of these distinct, locked positions. The number of serrations can also be modified to either increase or decrease the number of distinct interlocking positions of two adjacent cells 220 and 220a.

### Material Characteristics

Accordingly, in one exemplary embodiment, an intraluminal scaffold element may be fabricated from a non-metallic material such as a polymeric material including non-crosslinked thermoplastics, cross-linked thermosets, composites and blends thereof. There are typically three different forms in which a polymer may display the mechanical properties associated with solids; namely, as a crystalline structure, as a semi-crystalline structure and/or as an amorphous structure. All polymers are not able to fully crystallize, as a high degree of molecular regularity within the polymer chains is essential for crystallization to occur. Even in polymers that do crystallize, the degree of crystallinity is generally less than one hundred percent. Within the continuum between fully crystalline and amorphous structures, there are two thermal transitions possible; namely, the crystal-liquid transition (i.e. melting point temperature, Tₘ) and the glass-liquid transition (i.e. glass transition temperature, T_{g}). In the temperature range between these two transitions there may be a mixture of orderly arranged crystals and chaotic amorphous polymer domains.

The Hoffman-Lauritzen theory of the formation of polymer crystals with “folded” chains owes its origin to the discovery in 1957 that thin single crystals of polyethylene may be grown from dilute solutions. Folded chains are preferably required to form a substantially crystalline structure. Hoffman and Lauritzen established the foundation of the kinetic theory of polymer crystallization from “solution” and “melt” with particular attention to the thermodynamics associated with the formation of chain-folded nuclei.

Crystallization from dilute solutions is required to produce single crystals with macroscopic perfection (typically magnifications in the range of about 200× to about 400×). Polymers are not substantially different from low molecular weight compounds such as inorganic salts in this regard. Crystallization conditions such as temperature, solvent and solute concentration may influence crystal formation and final form. Polymers crystallize in the form of thin plates or “lamellae.” The thickness of these lamellae is on the order of ten nanometers (10 nm). The dimensions of the crystal plates perpendicular to the small dimensions depend on the conditions of the crystallization but are many times larger than the thickness of the platelets for a well-developed crystal. The chain direction within the crystal is along the short dimension of the crystal, which indicates that, the molecule folds back and forth (e.g. like a folded fire hose) with successive layers of folded molecules resulting in the lateral growth of the platelets. A crystal does not consist of a single molecule nor does a molecule reside exclusively in a single crystal. The loop formed by the chain as it emerges from the crystal turns around and reenters the crystal. The portion linking the two crystalline sections may be considered amorphous polymer. In addition, polymer chain ends disrupt the orderly fold patterns of the crystal, as described above, and tend to be excluded from the crystal. Accordingly, the polymer chain ends become the amorphous portion of the polymer. Therefore, no currently known polymeric material may be one-hundred percent crystalline. Post polymerization processing conditions dictate the crystal structure to a substantial extent.

Single crystals are not observed in crystallization from bulk processing. Bulk crystallized polymers from melt exhibits domains called “spherulites” that are symmetrical around a center of nucleation. The symmetry is perfectly circular if the development of the spherulite is not impinged by contact with another expanding spherulite. Chain folding is an essential feature of the crystallization of polymers from the molten state. Spherulites are comprised of aggregates of “lamellar” crystals radiating from a nucleating site. Accordingly, there is a relationship between solution and bulk grown crystals.

The spherical symmetry develops with time. Fibrous or lathlike crystals begin branching and fanning out as in dendritic growth. As the lamellae spread out dimensionally from the nucleus, branching of the crystallites continue to generate the spherical morphology. Growth is accomplished by the addition of successive layers of chains to the ends of the radiating laths. The chain structure of polymer molecules suggests that a given molecule may become involved in more than one lamella and thus link radiating crystallites from the same or adjacent spherulites. These interlamellar links are not possible in spherulites of low molecular weight compounds, which show poorer mechanical strength as a consequence.

The molecular chain folding is the origin of the “Maltese” cross, which identifies the spherulite under crossed polarizers. For a given polymer system, the crystal size distribution is influenced by the initial nucleation density, the nucleation rate, the rate of crystal growth, and the state of orientation. When the polymer is subjected to conditions in which nucleation predominates over radial growth, smaller crystals result. Larger crystals will form when there are relatively fewer nucleation sites and faster growth rates. The diameters of the spherulites may range from about a few microns to about a few hundred microns depending on the polymer system and the crystallization conditions.

Therefore, spherulite morphology in a bulk-crystallized polymer involves ordering at different levels of organization; namely, individual molecules folded into crystallites that in turn are oriented into spherical aggregates. Spherulites have been observed in organic and inorganic systems of synthetic, biological, and geological origin including moon rocks and are therefore not unique to polymers.

Stress induced crystallinity is important in film and fiber technology. When dilute solutions of polymers are stirred rapidly, unusual structures develop which are described as having a “shish kebab” morphology. These consist of chunks of folded chain crystals strung out along a fibrous central column. In both the “shish” and the “kebab” portions of the structure, the polymer chains are parallel to the overall axis of the structure.

When a polymer melt is sheared and quenched to a thermally stable condition, the polymer chains are perturbed from their random coils to easily elongate parallel to the shear direction. This may lead to the formation of small crystal aggregates from deformed spherulites. Other morphological changes may occur, including spherulite to fibril transformation, polymorphic crystal formation change, reorientation of already formed crystalline lamellae, formation of oriented crystallites, orientation of amorphous polymer chains and/or combinations thereof.

Molecular orientation is important as it primarily influences bulk polymer properties and therefore will have a strong effect on the final properties that are essential for different material applications. Physical and mechanical properties such as permeability, wear, refractive index, absorption, degradation rates, tensile strength, yield stress, tear strength, modulus and elongation at break are some of the properties that will be influenced by orientation. Orientation is not always favorable as it promotes anisotropic behavior. Orientation may occur in several directions such as uniaxial, biaxial and multiaxial. It may be induced by drawing, rolling, calendaring, spinning, blowing, and any other suitable process, and is present in systems including fibers, films, tubes, bottles, molded and extruded articles, coatings, and composites. When a polymeric material is processed, there will be preferential orientation in a specific direction. Usually it is in the direction in which the process is conducted and is called the machine direction (MD). Many of the products are purposely oriented to provide improved properties in a particular direction. If a product is melt processed, it will have some degree of preferential orientation. In case of solvent processed materials, orientation may be induced during processing by methods such as shearing the polymer solution followed by immediate precipitation or quenching to the desired geometry in order to lock in the orientation during the shearing process. Alternately, if the polymers have rigid rod like chemical structure then it will orient during processing due to the liquid crystalline morphology in the polymer solution.

The orientation state will depend on the type of deformation and the type of polymer. Even though a material is highly deformed or drawn, it is not necessary to impart high levels of orientation as the polymer chains may relax back to their original state. This generally occurs in polymers that are very flexible at the draw temperature. Therefore, several factors may influence the state of orientation in a given polymer system, including rate of deformation for example, strain rate, shear rate, frequency, and the like, amount of deformation or draw ratio, temperature, molecular weight and its distribution, chain configuration for example, stereoregularity, geometrical isomers, and the like, chain architecture, for example, linear, branched, cross-linked, dendritic and the like, chain stiffness, for example, flexible, rigid, semi-rigid, and the like, polymer blends, copolymer types, for example, random, block, alternating, and the like, and the presence of additives, for example, plasticizers, hard and soft fillers, long and short fibers, therapeutic agents and the like.

Since polymers consist of two phases; namely, crystalline and amorphous, the effect of orientation will differ for these phases, and therefore the final orientation may not be the same for these two phases in a semi-crystalline polymer system. This is because the flexible amorphous chains will respond differently to the deformation and the loading conditions than the hard crystalline phase.

Different phases may be formed after inducing orientation and its behavior depends on the chemistry of the polymer backbone. A homogenous state such as a completely amorphous material would have a single orientation behavior. However, in polymers that are semi-crystalline, block co-polymers or composites, for example, fiber reinforced, filled systems and liquid crystals, the orientation behavior needs to be described by more than one parameter. Orientation behavior, in general, is directly proportional to the material structure and orientation conditions. There are several common levels of structure that exist in a polymeric system, such as crystalline unit cell, lamellar thickness, domain size, spherulitic structures, oriented superstructures, phase separated domains in polymer blends and the like.

For example, in extruded polyethylene, the structure is a stacked folded chain lamellar structure. The orientation of the lamellae within the structure is along the machine direction, however the platelets are oriented perpendicular to the machine direction. The amorphous structure between the lamellae is generally not oriented. Mechanical properties of the material will be different when tested in different directions, for example, zero degree to the machine direction, forty-five degrees to the machine direction and ninety degrees to the machine direction. The elongation values are usually lowest when the material is stretched in machine direction. When stretched at forty-five degrees to the machine direction, shear deformation occurs of the lamellae and will provide higher elongation values. When stretched at ninety degrees to the machine direction, the material will exhibit highest elongation as the chain axis is unfolding.

When a polymer chain is oriented at an angle with respect to a given deformation axis, the orientation of the chain may be defined by Hermans orientation function, f, which varies from 1, −½ and 0 representing perfect orientation, perpendicular orientation, and random orientation along the axis, respectively. This applies mainly to uniaxially oriented systems. There are several techniques used to measure orientation such as birefringence, linear dichroism, wide angle x-ray scattering, polarized Raman scattering, polarized fluorescence, and nuclear magnetic resonance imaging or NMR.

### Process

According to the systems and methods of the present invention, a drug delivery device comprised of polymeric, bioabsorbable materials may be made by any of a variety of processes. The processes used to prepare the drug delivery devices are preferably low temperature processes in order to minimize the degradation of drugs or other bio-active agents that are unstable at high temperatures and are incorporated into the matrix of bioabsorbable polymeric materials comprising the device. Processing methods may comprise forming the device from bioabsorbable polymeric materials via low temperature, solution-based processes using solvents as by, for example, fiber spinning, including dry and wet spinning, electrostatic fiber spinning, co-mingled fibers, solvent extraction, coating, wire-coating, hollow fiber and membrane spinning, spinning disk (thin films with uniform thickness), ink-jet printing (three dimensional printing and the like), lyophilization, extrusion and co-extrusion, supercritical fluids, solvent cast films, or solvent cast tubes. Alternately, the drug delivery devices may also be prepared by more conventional polymer processing methods in melt condition for drugs or agents that are stable at high temperature as by, for example, fiber spinning, extrusion, co-extrusion, injection molding, blow molding, pultrusion and compression molding. Alternately, drugs may also be incorporated in the drug delivery device by diffusion through the polymer matrix. This may be achieved by several methods such as swelling the device in a drug-enriched solution followed by high-pressure diffusion or by swelling and diffusing the drug in the device using supercritical fluids. Alternately, the drugs or agents may be sprayed, dipped, or coated onto the device after formation thereof from the bioabsorbable polymers. In either case, the polymer matrix, and drug or agent blend when provided, is then converted into a structure such as fibers, films, discs/rings or tubes, for example, that is thereafter further manipulated into various geometries or configurations as desired.

Different processes may provide different structures, geometries or configurations to the bioabsorbable polymer being processed. For example, tubes processed from rigid polymers tend to be very stiff, but may be very flexible when processed via electrostatic processing or lyophilization. In the former case, the tubes are solid, whereas in the latter case, the tubes are porous. Other processes provide additional geometries and structures that may include fibers, microfibers, thin and thick films, discs, foams, microspheres and even more intricate geometries or configurations. Melt or solution spun fibers, films and tubes may be further processed into different designs such as tubular, slide and lock, helical or otherwise by braiding and/or laser cutting. The differences in structures, geometries or configurations provided by the different processes are useful for preparing different drug delivery devices with desired dimensions, strengths, drug delivery and visualization characteristics. The fibers, films or tubes may be laser cut to a desired geometry or configuration such as in the shape of a stent. Other machining techniques may also be utilized

Different processes may likewise alter the morphological characteristics of the bioabsorbable polymer being processed. For example, when dilute solutions of polymers are stirred rapidly, the polymers tend to exhibit polymer chains that are generally parallel to the overall axis of the structure. On the other hand, when a polymer solution or melt is sheared and quenched to a thermally stable condition, the polymer chains tend to elongate parallel to the shear direction. Still other morphological changes tend to occur according to other processing techniques. Such changes may include, for example, spherulite to fibril transformation, polymorphic crystal formation change, re-orientation of already formed crystalline lamellae, formation of oriented crystallites, orientation of amorphous polymer chains, crystallization, and/or combinations thereof.

In the case of a stent comprised of bioabsorbable polymeric materials formed by supercritical fluids, such as supercritical carbon dioxide, the supercritical fluids are used to lower processing temperatures during extrusion, molding or otherwise conventional processing techniques. Different structures, such as fibers, tubes, films, or foams, may be formed using the supercritical fluids, whereby the lower temperature processing that accompanies the supercritical fluids tends to minimize degradation of the drugs incorporated into the structures formed.

### Solvent Processing

In the case of a stent comprised of bioabsorbable polymeric materials formed by tubes from solution, the viscosity of the polymer solution will determine the processing method used to prepare the tubes. Viscosity of the polymer solutions will, in turn, depend on factors such as the molecular weight of the polymer, polymer concentration, the solvent used to prepare the solutions, processing temperatures and shear rates. Polymers with relatively high molecular weight, for example, an average molecular weight above 300,000 Daltons and an intrinsic viscosity above 2.0 dl/g, have been used in accordance with the present invention.

Polymer solutions (approximately 1 percent to 20 percent (wt/wt) concentration), for example, prepared from PLGA with an intrinsic viscosity of 2 to 2.5 dl/g in dioxane comprising a drug in the range from about 0 percent to about 50 percent may be directly deposited on a mandrel using a needle, for example, at room temperature or at temperatures that will not degrade the drug, using a syringe pump. Alternately, mandrels may be dip coated in the solutions followed by drying and subsequent dip coating steps to obtain the required wall thickness. Different mandrel sizes may be used to obtain varying final tube dimensions, for example, diameter, wall thickness and the like. Process optimization such as solution flow rate, mandrel RPM, traverse speed and the size of the needle may be implemented to obtain high quality tubes with uniform diameter and wall thickness that will be suitable to prepare stents. The polymer solutions may also contain radiopaque agents and other additives such as plasticizers, other polymers, and the like. The solvent from the drug loaded polymer tube on the mandrel may then be removed at temperatures and conditions that will not degrade the drug. For example, thermal and/or vacuum drying, supercritical carbon dioxide, lyophilization and combinations thereof. The tubes may then be converted into stents, for example, by laser cutting or any other suitable machining techniques.

Polymer solutions (approximately 20 percent to 50 percent (wt/wt) concentration), for example, prepared from PLGA with an intrinsic viscosity of 2 to 2.5 dl/g in dioxane comprising a drug in the range from about 0 percent to about 50 percent may be extruded vertically through an annular die using a gear pump and by passing it through a hot chimney to evaporate the solvent to form a tube. Alternately, the polymer solution may be extruded horizontally through an annular die using a gear pump and by passing it through a non-solvent, water bath, for example, to precipitate the solution to form a tube. The hollow tube extruded vertically or horizontally may then be collected on a take-up device or a wheel that will not crush the tube and will retain the shape. Alternately, the lumen of the die may have a metallic mandrel or monofilament fiber or pressurized gas and/or air to prevent the tube from collapsing during the extrusion process. The solvent from the drug loaded polymer tube may then be removed at temperatures and conditions that will not degrade the drug. Process optimization such as solution flow rate, solution temperature, take up speed, air and coagulation temperature may be implemented to obtain high quality tubes with uniform diameter and wall thickness that will be suitable to prepare stents. The polymer solutions may also contain radiopaque agents and other additives such as plasticizers, other polymers and the like.

Another method to prepare tubes from polymer solutions, for example in the range from about 1 percent to 50 percent (wt/wt), is to extrude the solutions using an extruder with a tubular die. During extrusion, the viscosity of the solution may be raised by gradual removal or multi-stage de-volatilization of solvent from vents using, for example, vacuum pumps. Twin screw or vented screw extruders may be used for this purpose. Residual solvent may be further removed at temperatures and conditions that will not degrade the drug. The polymer solutions may also comprise radiopaque agents and other additives such as plasticizers, other polymers and the like.

When the concentration of polymer in the solvent becomes higher than a certain value, it transitions to form extremely viscous solutions, gels or swollen networks. These systems may be prepared by mixing with or exposing the polymer to the solvent or plasticizer and drug to form a uniformly distributed formulation. Different mixing methods may be used to prepare the formulations such as for example, high shear low temperature mixers, for example, the Henschel Mixer, and counter or co-rotating twin-screw extruders at low temperature using different elements such as high shear mixing and kneading elements. After mixing the components, the mixture may be allowed to equilibrate so that the solvent or plasticizer is well distributed in and around the polymer resin. In order to prevent any solvent loss, the mixture is tightly enclosed in a jar or other suitable container and stored at temperature that will prevent re-crystallization, agglomeration, and solvent evaporation. These equilibrated mixtures may then be extruded vertically or horizontally, for example, using a high-pressure gear pump and a tubular die at low temperatures that will not degrade the drug, and will not evaporate the solvent. Maintaining consistent solvent levels during extrusion is critical so that the material is processed uniformly in the barrel without any variations in viscosity. This may be achieved by using conventional melt extrusion technology. Alternately, billets may be formed from the formulation and can be extruded by ram extrusion to prepare tubes. Other methods that are used to process gels and swollen materials can also be adapted to prepare tubes. Examples include materials such as polytetrafluoroethylene and ultrahigh molecular weight polyethylene. The solvent may be removed during and after extrusion as described by the methods above.

For example, polymer formulation approximately above 50 percent (wt/wt) concentration, prepared from PLGA with an intrinsic viscosity of 2 to 2.5 dl/g in dioxane comprising a drug in the range from about 0 percent to about 50 percent may be extruded using a high-pressure gear pump and a tubular die. The extrusion will be conducted at temperatures that will not degrade the drug and in a relatively short residence time in the barrel. The solvent from the drug loaded polymer tube may then be removed at temperatures and conditions that will not degrade the drug. The polymer formulations may also comprise radiopaque agents and other additives such as plasticizers, other polymers and the like.

All the solvent processed tubes may be prepared in different shapes, geometries and configurations. For example, the tube may be co-extruded and/or wire coated. Other processing methodologies that are known in the art may be utilized.

The amount of solvent or plasticizer required to process the materials at low temperatures will depend on the polymer morphology. It may require lesser amounts of solvent or plasticizer to achieve low temperature processing conditions for amorphous material compared to semi-crystalline materials. This is because amorphous phase is relatively easier to dissolve or swell compared to the crystalline phase. In order to obtain a homogenous morphology, the polymer may be melt extruded at high temperature (above its melting point) followed by quenching to form an amorphous material. This amorphous material may then be used to mix with the solvent or plasticizer to achieve low temperature processing conditions as described above. In general, the greater the amount of solvent or plasticizer, the lower the melt temperature and the lower the melt viscosity of the blend.

### Melt Processing

Drug delivery devices as well as non-drug delivery devices may also be prepared by more conventional polymer processing methods in melt condition for drugs or agents that are stable at high temperature. Melt process may also be used for drug delivery devices in which the polymers are not readily soluble in solvents. Polymer compounding may be achieved by using twin-screw extruders with different screw elements to achieve desired mixing and dispersion. There are also feeders to add additives during the compounding process to from multi-component blends or composites. These additives may include pellets, powders of different sizes, short fibers or liquids. Polymer and drug, for example, 1 percent to about 50 percent (wt/wt) may be melt-compounded using a twin-screw extruder at low temperatures under low shear conditions. The compounded material may be pelletized and extruded into a tube of desired geometry (wall thickness, etc) using a single screw extruder. The tubes may then be laser cut to prepare a stent. As stated above, other machining techniques may be utilized. Radiopaque agents for example, from about 1 percent to about 40 percent (wt/wt) and other additives such as plasticizers and other polymers may also be added to the polymer formulation during the compounding step.

Polymers may be compounded with radiopaque agents or other polymers and plasticizers without the drug for temperature sensitive drug or agents as described herein. Melt processing temperatures may be raised sufficiently to achieve proper melting for proper compounding and tube extrusion; however, care should be taken to avoid degrading the polymers. Drugs may then be coated on the laser cut stent prepared from these materials. In this case, it is important to select solvents that will evaporate quickly and will not readily dissolve or swell the stent materials to prevent solvent penetration inside the stent that will cause buckling and stent deformation.

In the case of a stent device comprised of bioabsorbable materials formed by co-extrusion, different bioabsorbable polymeric materials may be used whereby the different polymer tubes or fibers are extruded generally at the same time to form an outer layer for tubes or sheaths in case of fibers, and a inner layer for tubes or core in case of fibers. Bioabsorbable polymeric materials having low melting points are extruded to form the sheath or outside surface, and these low melting point materials will incorporate the drugs or other bio-active agents for eventual delivery to the patient. Materials and their blends having higher melting points are extruded to form the core or inside surface that is surrounded by the sheath. The higher melting point materials comprising the core or inner surface will thus provide strength to the stent. During processing, the temperatures for extruding the low melting point drug comprising materials, for example, polycaprolactone, polydioxanone, and their copolymers and blends may be as low as 60 degrees C. to 100 degrees C. Further, because the drugs or other bio-active agents added to the devices made by this co-extrusion method tend to be coated onto the device after the device has been extruded, the drugs or agents are not exposed to the high temperatures associated with such methods. Degradation of the drugs during processing is therefore minimized. Radiopaque agents or other additives may be incorporated into the device during or after extrusion thereof.

In the case of a stent device comprised of bioabsorbable polymeric materials formed by co-mingled fibers, different bioabsorbable polymeric materials may also be used. Contrary to the co-extrusion techniques described above, the co-mingled fibers technique requires that each fiber be separately extruded and then later combined to form a stent of a desired geometry. Alternately, different fibers may also be extruded using the same spin pack but from different spinning holes thereby combining them in one step. The different bioabsorbable polymeric materials include a first fiber having a low temperature melting point into which a drug is incorporated, and a second fiber having a higher temperature melting point. As before, radiopaque agents and other additives such as polymers and plasticizers may be added to one or more of the fibers during, or after, extrusion thereof.

There are several different morphological variations that may occur during melt or solution processing bioabsorbable materials. When semi-crystalline polymers are processed from solution, since the solvent evaporates gradually, the polymers may get sufficient time to re-crystallize before it is completely dry. It will also allow time for phase separation to occur in case of multi-component blend systems. These changes are driven by well-known theories of thermodynamics of polymer crystallization and phase separation. In order to prepare, for example, amorphous tubes or films from solution, it may be necessary to remove the solvent in a relatively short time so that kinetics will prevent crystallization and phase separation from occurring. For example, when the PLGA tubes are prepared from dioxane solutions, it may be necessary to remove the solvent in a relatively short time, for example, a few minutes to hours at low temperatures, for example, below 60 degrees C., after the tube forming process to obtain an almost amorphous tube. If the solvent removal process is carried out over a long period of time, for example, 6 to 10 h, at elevated temperatures, for example, 60 degrees C., then PLGA may begin to crystallize (up to 10 to 20 percent crystallinity). In case of polymer blends, it is preferred to have an amorphous system to achieve good compatibility between the amorphous phases of the polymers so that the physical properties are not adversely affected. When the polymer solutions are precipitated or coagulated as described above in the hollow tube extrusion process, the resulting tube will be almost amorphous (1 to 5 percent crystallinity), as the solvent removal process is very fast thereby not allowing the polymer to crystallize.

In case of melt processed materials, the tubes or films are quenched immediately after exiting the extrusion die. Therefore, the polymers, in general, do not crystallize if the quenched temperature is below the glass transition temperature of the materials. In case of PLGA, the extruded tubes have very low levels of crystallinity (1 to 5 percent). This also makes it favorable when polymer blends are prepared from this process. Annealing the materials between the glass transition and melt temperatures for a given period of time will increase the amount of crystallinity. For example, PLGA tubes may be annealed at 110 degrees C. for 3 to 10 h by mounting them over a mandrel under tension to prevent any shrinkage or buckling. The amount of crystallinity will increase from about 0 percent to about 35 to 45 percent. Accordingly, this way the tube properties may be altered to achieve the desired morphology and physical properties.

These morphological variations in the precursor material (tubes, films, etc) will dictate to some extent the performance of the devices prepared from these materials. Some examples of stent performance factors include radial strength, recoil and flexibility. Amorphous materials will absorb faster, have higher toughness values, will physically age, and may not have sufficient dimensional stability compared to crystalline material. In contrast, crystalline material may not form compatible blends, will take a longer time to absorb, are stiffer with lower toughness values, and may have superior physical device properties such as low creep, higher radial strength, etc. For example, a material that is mechanically tested from a quenched state (higher amorphous form) and a slow cooled state (higher crystalline form) will provide a ductile high deformation behavior and a brittle behavior, respectively. This behavior is from the differences in the crystallinity and morphological features driven by different thermal treatments and histories. The morphological structure of a device surface may be modified by applying energy treatment (e.g., heat) to the abluminal and/or luminal surface. For example, an amorphous surface morphology can be converted to a crystalline surface morphology by annealing it under different conditions (temperature/time). This may result in the formation of a crystalline skin or layer on the device that may provide several benefits such as drug elution control and surface toughness to prevent crack formation and propagation. Therefore, it is important to balance the structure—property—processing relationship for the materials that are used to prepare the devices to obtain optimum performance.

The stents and/or other implantable medical devices of the current invention may be prepared from pure polymers, blends, and composites and may be used to prepare drug-loaded stents. The precursor material may be a tube or a film that is prepared by any of the processes described above, followed by laser cutting or any other suitable machining process. The precursor material may be used as prepared or can be modified by quenching, annealing, orienting or relaxing them under different conditions. Alternately, the laser cut stent may be used as prepared or may be modified by quenching, annealing, orienting or relaxing them under different conditions.

### Mechanical Orientation

The effect of polymer orientation in a stent or device may improve the device performance including radial strength, recoil, and flexibility. Orientation may also vary the degradation time of the stent, so as desired, different sections of the stents may be oriented differently. Orientation may be along the axial and circumferential or radial directions as well as any other direction in the unit cell and flex connectors to enhance the performance of the stent in those respective directions. The orientation may be confined to only one direction (uniaxial), may be in two directions (biaxial) and/or multiple directions (multiaxial). The orientation may be introduced in a given material in different sequences, such as first applying axial orientation followed by radial orientation and vice versa. Alternately, the material may be oriented in both directions at the same time. Axial orientation may be applied by stretching along an axial or longitudinal direction in a given material such as tubes or films at temperatures usually above the glass transition temperature of the polymer. Radial or circumferential orientation may be applied by several different methods such as blowing the material by heated gas for example, nitrogen, or by using a balloon inside a mold. Alternately, a composite or sandwich structure may be formed by stacking layers of oriented material in different directions to provide anisotropic properties. Blow molding may also be used to induce biaxial and/or multiaxial orientation.

Orientation may be imparted to tubes, films or other geometries that are loaded with drugs in the range from about 1 to 50 percent. For example, drug loaded PLGA tubes prepared by any of the above-mentioned processes may be oriented at about 70 degrees C. to different amounts (for example, 50 percent to 300 percent) at different draw rates (for example, 100 mm/min to 1000 mm/min). The conditions to draw the material is important to prevent excessive fibrillation and void formation that may occur due to the presence of drug. If the draw temperature is increased to a higher value (for example, 90 degrees C.), then the orientation may not be retained as the temperature of orientation is much higher than the glass transition temperature of PLGA (about 60 degrees C.) and would cause relaxation of the polymer chains upon cooling.

Other methods of orienting the materials may include multi-stage drawing processes in which the material or device may be drawn at different draw rates at different temperatures before or after intermediate controlled annealing and relaxation steps. This method allows increasing the total draw ratio for a given material that is not otherwise possible in one-step drawing due to limitations of the material to withstand high draw ratio. These steps of orientation, annealing and relaxation will improve the overall strength and toughness of the material.

### Polymeric Materials

Polymeric materials may be broadly classified as synthetic, natural and/or blends thereof. Within these broad classes, the materials may be defined as biostable or biodegradable. Examples of biostable polymers include polyolefins, polyamides, polyesters, fluoropolymers, and acrylics. Examples of natural polymers include polysaccharides and proteins.

The drug delivery devices according to the systems and methods of the present invention may be disease specific, and may be designed for local or regional therapy, or a combination thereof. They may be used to treat coronary and peripheral diseases such as vulnerable plaque, restenosis, bifurcated lesions, superficial femoral artery, below the knee, saphenous vein graft, arterial tree, small and tortuous vessels, and diffused lesions. The drugs or other agents delivered by the drug delivery devices according to the systems and methods of the present invention may be one or more drugs, bio-active agents such as growth factors or other agents, or combinations thereof. The drugs or other agents of the device are ideally controllably released from the device, wherein the rate of release depends on either or both of the degradation rates of the bioabsorbable polymers comprising the device and the nature of the drugs or other agents. The rate of release can thus vary from minutes to years as desired.

Bioabsorbable and/or biodegradable polymers consist of bulk and surface erodable materials. Surface erosion polymers are typically hydrophobic with water labile linkages. Hydrolysis tends to occur fast on the surface of such surface erosion polymers with no water penetration in bulk. The initial strength of such surface erosion polymers tends to be low however, and often such surface erosion polymers are not readily available commercially. Nevertheless, examples of surface erosion polymers include polyanhydrides such as poly(carboxyphenoxy hexane-sebacic acid), poly(fumaric acid-sebacic acid), poly(carboxyphenoxy hexane-sebacic acid), poly(imide-sebacic acid) (50-50), poly(imide-carboxyphenoxy hexane) (33-67), and polyorthoesters (diketene acetal based polymers).

Bulk erosion polymers, on the other hand, are typically hydrophilic with water labile linkages. Hydrolysis of bulk erosion polymers tends to occur at more uniform rates across the polymer matrix of the device. Bulk erosion polymers exhibit superior initial strength and are readily available commercially.

Examples of bulk erosion polymers include poly(α-hydroxy esters) such as poly(lactic acid), poly(glycolic acid), poly(caprolactone), poly(p-dioxanone), poly(trimethylene carbonate), poly(oxaesters), poly(oxaamides), and their co-polymers and blends. Some commercially readily available bulk erosion polymers and their commonly associated medical applications include poly(dioxanone) [PDS® suture available from Ethicon, Inc., Somerville, N.J.], poly(glycolide) [Dexon® sutures available from United States Surgical Corporation, North Haven, Conn.], poly(lactide)-PLLA [bone repair], poly(lactide/glycolide) [Vicryl® (10/90) and Panacryl® (95/5) sutures available from Ethicon, Inc., Somerville, N.J.], poly(glycolide/caprolactone (75/25) [Monocryl® sutures available from Ethicon, Inc., Somerville, N.J.], and poly(glycolide/trimethylene carbonate) [Maxon® sutures available from United States Surgical Corporation, North Haven, Conn.].

Other bulk erosion polymers are tyrosine derived poly amino acid [examples: poly(DTH carbonates), poly(arylates), and poly(imino-carbonates)], phosphorous containing polymers [examples: poly(phosphoesters) and poly(phosphazenes)], poly(ethylene glycol) [PEG] based block co-polymers [PEG-PLA, PEG-poly(propylene glycol), PEG-poly(butylene terephthalate)], poly(α-malic acid), poly(ester amide), and polyalkanoates [examples: poly(hydroxybutyrate (HB) and poly(hydroxyvalerate) (HV) co-polymers].

Of course, the devices may be made from combinations of surface and bulk erosion polymers in order to achieve desired physical properties and to control the degradation mechanism. For example, two or more polymers may be blended in order to achieve desired physical properties and device degradation rate. Alternately, the device may be made from a bulk erosion polymer that is coated with a surface erosion polymer. The drug delivery device may be made from a bulk erosion polymer that is coated with a drug containing a surface erosion polymer. For example, the drug coating may be sufficiently thick that high drug loads may be achieved, and the bulk erosion polymer may be made sufficiently thick that the mechanical properties of the device are maintained even after all of the drug has been delivered and the surface eroded.

Shape memory polymers may also be used. Shape memory polymers are characterized as phase segregated linear block co-polymers having a hard segment and a soft segment. The hard segment is typically crystalline with a defined melting point, and the soft segment is typically amorphous with a defined glass transition temperature. The transition temperature of the soft segment is substantially less than the transition temperature of the hard segment in shape memory polymers. A shape in the shape memory polymer is memorized in the hard and soft segments of the shape memory polymer by heating and cooling techniques. Shape memory polymers may be biostable and bioabsorbable. Bioabsorbable shape memory polymers are relatively new and comprise thermoplastic and thermoset materials. Shape memory thermoset materials may include poly(caprolactone) dimethylacrylates, and shape memory thermoplastic materials may include poly(caprolactone) as the soft segment and poly(glycolide) as the hard segment.

The selection of the bioabsorbable polymeric material used to comprise the drug delivery device according to the invention is determined according to many factors including, for example, the desired absorption times and physical properties of the bioabsorbable materials, and the geometry of the drug delivery device.

### Properties/Blends

Toughness of a system is the mechanical energy or work required to induce failure, and depends on testing conditions such as temperatures and loading rates. Toughness is the area under the engineering stress-strain curve, and is therefore an ultimate property for a given material. For this reason, it is important to obtain data from a large population of specimens in order to achieve accurate toughness values. Toughness of polymers may fall in to several different categories. A material that is hard and brittle will have high modulus and low strain at break values and will therefore have low toughness, and a material that is hard and tough will have high modulus and high strain at break values and will therefore have high toughness. Similarly, a material that is soft and weak will have low modulus and low strain at break values and will have low toughness, and a material that is soft and tough will have low modulus and high strain at break values and will have high toughness values. Ideally, it is desirable to have a material with high toughness that has high modulus and high strain at break or ultimate strain values for a vascular device such as drug loaded stent.

Mechanical hysteresis is the energy that is lost during cyclic deformation, and is an important factor in dynamic loading applications of polymers such as in vascular stents. Since polymers are viscoelastic materials, they all exhibit mechanical hysteresis unlike in elastic materials where there is no energy loss during cyclic deformation. The amount or percent of mechanical hysteresis depends on the type of polymers. For example, it is possible that elastomers will have low percent mechanical hysteresis compared to a stiff and brittle non-elastomeric material. Also, non-elastomeric materials may also have permanent set after removing load from its deformed state.

In order to provide materials with high toughness, such as is often required for orthopedic implants, sutures, stents, grafts and other medical applications including drug delivery devices, the bioabsorbable polymeric materials may be modified to form composites or blends thereof. Such composites or blends may be achieved by changing either the chemical structure of the polymer backbone, or by creating composite structures by blending them with different polymers and plasticizers.

The addition of plasticizers, which are generally low molecular weight materials, or a soft (lower glass transition temperature) miscible polymer, will depress the glass transition temperature of the matrix polymer system. In general, these additional materials that are used to modify the underlying bioabsorbable polymer should preferably be miscible with the main matrix polymer system to be effective.

In accordance with the present invention, the matching of a suitable polymer or blends thereof and plasticizer or mixtures thereof to form a blend for the preparation of a drug loaded stent or device, or a stent or device with no drug is important in achieving desirable properties. Combining the polymers and plasticizers is accomplished by matching the solubility parameters of the polymer component and plasticizer component within a desired range. Solubility parameters of various materials and methods of calculating the same are known in the art. The total solubility parameter of a compound is the sum of the solubility parameter values contributed by dispersive forces, hydrogen bonding forces and polar forces. A polymer will dissolve in a plasticizer or be plasticized if either the total solubility parameter or one or more of the disperse forces, polar forces, and hydrogen bonding forces for each of the polymer and plasticizer are similar.

Free volume is the space between molecules, and it increases with increased molecular motion. Accordingly, a disproportionate amount of free volume is associated with chain end groups in a polymer system. Increasing the concentration of chain end groups increases the free volume. The addition of flexible side chains in to macromolecules therefore increases the free volume. All of these effects may be used for internal plasticization, and free volume is spatially fixed with regard to the polymer molecule. However, the addition of a small molecule affects the free volume of large macromolecules at any location by the amount of material added, which is known as external plasticization. The size and shape of the molecule that is added and the nature of its atoms and groups of atoms (i.e., non-polar, polar, hydrogen bonding, etc) determine how it functions as a plasticizer. The normal effect of increasing the free volume of a polymer is that it is plasticized (i.e., the glass transition temperature is lowered, the modulus and tensile strength decreases, and elongation at break and toughness increases). However, the freedom of movement afforded by the plasticizer also permits the polymer molecules to associate tightly with each other. In general, free volume is based on the principle that a suitable plasticizer increases the free volume of the polymer. An increase in free volume of the polymer increases the mobility of the polymer and therefore extent of plasticization. Thus, if more plasticization is desired, the amount of the plasticizer may be increased.

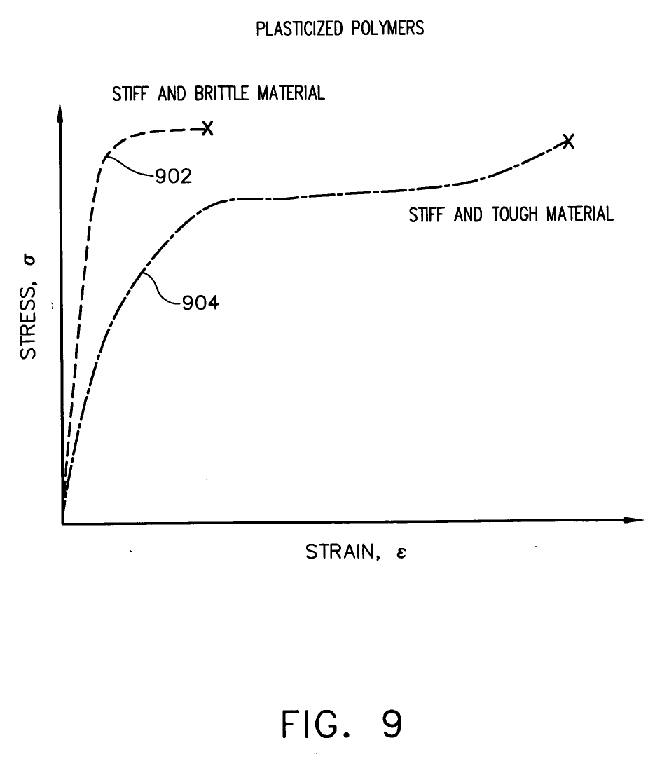

FIG. 9 is a schematic representation of the stress-strain behavior of a plasticized stiff and brittle material, represented by curve 904. The stiff and brittle polymeric material, represented by curve 902, is altered by the addition of a plasticizer. Stiff material has a higher modulus and low strain at break values with low toughness as the area under the curve is small. The addition of a plasticizer makes the stiff and brittle material a stiff and tough material. In other words, the addition of a plasticizer will lower the modulus to some extent but will increase the ultimate strain value thereby making the plasticized material tougher. As stated above, curve 904 represents the blend of a stiff and brittle polymer with a plasticizer resulting in a material with a modified stress-strain curve. The amount of change in modulus and toughness depends on the amount of plasticizer in the polymer. In general, the higher the amount of plasticizer, the lower the modulus and the higher the toughness values.

Plasticizers that are added to the matrix of bioabsorbable polymer materials will make the device more flexible and typically reduces the processing temperatures in case of processing materials in melt. The plasticizers are added to the bioabsorbable materials of the device prior to or during processing thereof. As a result, degradation of drugs incorporated into the bioabsorbable materials having plasticizers added thereto during processing is further minimized.

Plasticizers or mixtures thereof suitable for use in the present invention may be selected from a variety of materials including organic plasticizers and those like water that do not contain organic compounds. Organic plasticizers include but not limited to, phthalate derivatives such as dimethyl, diethyl and dibutyl phthalate; polyethylene glycols with molecular weights preferably from about 200 to 6,000, glycerol, glycols such as polypropylene, propylene, polyethylene and ethylene glycol; citrate esters such as tributyl, triethyl, triacetyl, acetyl triethyl, and acetyl tributyl citrates, surfactants such as sodium dodecyl sulfate and polyoxymethylene (20) sorbitan and polyoxyethylene (20) sorbitan monooleate, organic solvents such as 1,4-dioxane, chloroform, ethanol and isopropyl alcohol and their mixtures with other solvents such as acetone and ethyl acetate, organic acids such as acetic acid and lactic acids and their alkyl esters, bulk sweeteners such as sorbitol, mannitol, xylitol and lycasin, fats/oils such as vegetable oil, seed oil and castor oil, acetylated monoglyceride, triacetin, sucrose esters, or mixtures thereof. Preferred organic plasticizers include citrate esters; polyethylene glycols and dioxane.

Citrate esters are renewable resource derivatives derived from citric acid, a tribasic monohydroxy acid (2-hydroxy-1,2,3-propanetricarboxylic acid), C₆H₈O₇, and a natural constituent and common metabolite of plants and animals. They are non-toxic and have been used as plasticizers with a variety of different polymers. Different grades of citrate esters are available from Morflex, Inc. Typical molecular weights, boiling points, solubility in water and solubility parameters are 270 to 400 g/mole; 125 to 175 degrees C.; <0.1 to 6.5 g/100 mL and 18 to 20 (J/cm³)^{1/2}, respectively. Molecular weight has a strong influence on all the properties. As it increases, boiling point increases and the molecule becomes less polar as the water solubility and solubility parameters decreases.

Polyethylene glycols are water-soluble and are available in molecular weights ranging from 200 to 20,000 g/mole. The solubility decreases with increasing molecular weight. These materials are also soluble in polar organic solvents such as chloroform and acetone. These polymers are readily available from several suppliers.

Solubility parameter value of solvents such as dioxane and chloroform is about 20 and 19 MPa^{1/2}, respectively, and these are considered as some of the good solvents for bioabsorbable materials such as poly(lactic acid-co-glycolic acid). So, it may be assumed that the solubility parameter for these materials should be close to those of the solvents.

Citrate ester plasticizers may be added to bioabsorbable polymers in solution or in melt states from 1 to 50 percent, preferably from 1 to 35 percent and more preferably from 1 to 20 percent by weight in the presence of drug and/or radiopaque agent. The polymers may be selected from poly(lactic acid-co-glycolic acid) (95/5 to 85/15 ratio), the radiopaque agent is barium sulfate (preferred range is 10 percent to 50 percent) and the drug is sirolimus (preferred range is 1 percent to 30 percent). These may be converted to tubes or films from any of the processes described above. The elongation at break values for the polymer system increases to above 20 percent with the addition of 1 to 20 percent of the plasticizer. This exhibits significant increase in toughness and is very favorable for high strain balloon expandable stent designs.

Polymer blends are commonly prepared to achieve the desired final polymer properties. In accordance with the present invention, polymer blends are prepared to increase the elongation at break values or ultimate strain and thereby improving the toughness of the material that will be used to prepare vascular devices such as stents. Selection of the materials is important in order to achieve high toughness values of the matrix polymer. Matching solubility parameters and increase in free volume is important for the polymer blends to achieve the desired performance. The main difference between adding a plasticizer and a polymer to the matrix polymer is the difference in their molecular weights. As mentioned earlier, plasticizers have lower molecular weight compared to a polymeric additive. However, some low molecular weight polymers may also be used as a plasticizer. It is possible to achieve high toughness values by adding low amounts of plasticizer compared to a polymeric additive. Relatively high molecular weight material has been used as the matrix material for the present invention. For example, the molecular weight (weight average) of PLGA resins may be above 300,000 Daltons. Thermodynamically, molecular weight plays a big role in miscibility of polymer systems. There is higher miscibility between polymer and a low molecular weight additive compared to a high molecular weight additive. As mentioned earlier, the addition of a miscible polymer will lower glass transition temperature, decrease modulus and tensile strength with an increase in the toughness values.

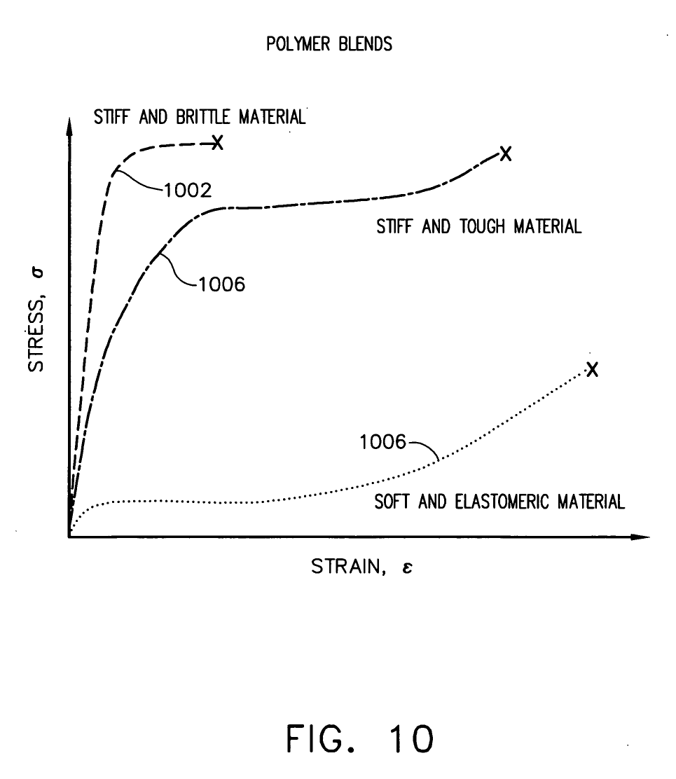

FIG. 10 is a schematic representation of the stress-strain behavior of a stiff and brittle material with high modulus and low strain at break values, i.e., low toughness, as represented by curve 1002 with a soft and elastomeric material with low modulus and relatively high strain at break values, as represented by curve 1004 and the resultant polymer blend prepared from these two materials, as represented by curve 1006, that will provide a relatively stiff material with high ultimate strain values, i.e., high toughness. The amount of change in modulus, strength and strain at break values depends on the amount of the polymeric additive in the matrix polymer. In general, the polymers are miscible or compatible at lower levels of the additive (for example <50 percent by weight) beyond which they become phase separated and the physical properties may begin to deteriorate. However, it is important to note that it is possible to achieve desirable compatibility between the phase separated polymers through the addition of bioabsorbable compatibilizers.

As an example of producing a composite or blended material, blending a stiff polymer such as poly(lactic acid), poly(glycolide) and poly(lactide-co-glycolide) copolymers with a soft and elastomeric polymer such as poly(caprolactone) and poly(dioxanone) tends to produce a material with high toughness and high stiffness. An elastomeric co-polymer may also be synthesized from a stiff polymer and a soft polymer in different ratios. For example, poly(glycolide) or poly(lactide) may be copolymerized with poly(caprolactone) or poly(dioxanone) to prepare poly(glycolide-co-caprolactone) or poly(glycolide-co-dioxanone) and poly(lactide-co-caprolactone) or poly(lactide-co-dioxanone) copolymers. These elastomeric copolymers may then be blended with stiff materials such as poly(lactide), poly(glycolide) and poly(lactide-co-glycolide) copolymers to produce a material with high toughness and ductility. Alternatively, terpolymers may also be prepared from different monomers to achieve desired properties. For example, poly(caprolactone-co-glycolide-co-lactide) may be prepared in different ratios.

Preferred materials for the matrix polymer are poly(lactic acid-co-glycolic acid) (95/5 and 85/15), which are usually stiff and brittle. Preferred soft and elastomeric materials for the polymers that are added to the matrix polymer are poly(caprolactone); poly(dioxanone); copolymers of poly(caprolactone) and poly(dioxanone); and co-polymers of poly(caprolactone) and poly(glycolide). The ratios of the monomer content for the copolymers may range from about 95/5 to about 5/95. Preferably, the ratios are about 95/5 to about 50/50 for poly(caprolactone)/poly(dioxanone) copolymer, and from about 25/75 to about 75/25 for poly(caprolactone)/poly(glycolide) copolymers. The addition of these polymers to the matrix polymer may vary from 1 percent to 50 percent, and more preferably from 5 to 35 percent (wt/wt). These blends should preferably comprise a high amount of drug (1 to 30 percent) such as sirolimus and radiopaque agents (10 to 50 percent) such as barium sulfate, and may be prepared using melt or solvent-based processes.

In addition to increasing the toughness values with the addition of the soft polymers, the absorption time may also be modified. For example, the blend of PLGA with polycaprolactone will increase the total absorption time of the blended material as polycaprolactone degrades slower than PLGA. The total absorption may be reduced for PLGA by blending it with faster degrading materials such as poly(dioxanone) and their copolymers with poly(glycolide) and poly(lactide); and copolymers of poly(glycolide) such as poly(caprolactone-co-glycolide).

Reinforced composites may also be prepared by blending high modulus PGA fibers or bioabsorbable particulate fillers with PLGA to form composites in the presence of the plasticizers or soft materials to improve the modulus of the final material.

Melt blends of polymers, with melting points lower than the melting point of the bioabsorbable materials in which the drugs or other bio-active agents are to be incorporated, may also be added to the bioabsorbable materials that are to comprise the device. Adding the blends of polymers having the lower melting points also helps to reduce processing temperatures and minimize degradation of the drugs or agents thereby.

It is important to note that the drug or therapeutic agent, in sufficient concentration, may be used as an additive for modifying the polymer properties. In other words, the drug or therapeutic agent may be utilized as part of the blend, rather than as a material affixed to a base material, similar to the blends described herein to achieve the desired end product properties in addition to providing a therapeutic effect.

### Additives

Because visualization of the device as it is implanted in the patient is important to the medical practitioner for locating the device, radiopaque materials may be added to the device. The radiopaque materials may be added directly to the matrix of bioabsorbable materials comprising the device during processing thereof resulting in fairly uniform incorporation of the radiopaque materials throughout the device. Alternately, the radiopaque materials may be added to the device in the form of a layer, a coating, a band or powder at designated portions of the device depending on the geometry of the device and the process used to form the device. Coatings may be applied to the device in a variety of processes known in the art such as, for example, chemical vapor deposition (CVD), physical vapor deposition (PVD), electroplating, high-vacuum deposition process, microfusion, spray coating, dip coating, electrostatic coating, or other surface coating or modification techniques. Such coatings sometimes have less negative impact on the physical characteristics (e.g., size, weight, stiffness, flexibility) and performance of the device than do other techniques. Preferably, the radiopaque material does not add significant stiffness to the device so that the device may readily traverse the anatomy within which it is deployed. The radiopaque material should be biocompatible with the tissue within which the device is deployed. Such biocompatibility minimizes the likelihood of undesirable tissue reactions with the device. Inert noble metals such as gold, platinum, iridium, palladium, and rhodium are well-recognized biocompatible radiopaque materials. Other radiopaque materials include barium sulfate (BaSO₄), bismuth subcarbonate [(BiO)₂CO₃] and bismuth oxide. Preferably, the radiopaque materials adhere well to the device such that peeling or delamination of the radiopaque material from the device is minimized, or ideally does not occur. Where the radiopaque materials are added to the device as metal bands, the metal bands may be crimped at designated sections of the device. Alternately, designated sections of the device may be coated with a radiopaque metal powder, whereas other portions of the device are free from the metal powder.

The bioabsorbable polymer materials comprising the drug delivery device according to the invention may include radiopaque additives added directly thereto during processing of the matrix of the bioabsorbable polymer materials to enhance the radiopacity of the device. The radiopaque additives may include inorganic fillers, such as barium sulfate, bismuth subcarbonate, bismuth oxides and/or iodine compounds. The radiopaque additives may instead include metal powders such as tantalum, tungsten or gold, or metal alloys having gold, platinum, iridium, palladium, rhodium, a combination thereof, or other materials known in the art. The particle size of the radiopaque materials may range from nanometers to microns, preferably from less than or equal to about 1 micron to about 5 microns, and the amount of radiopaque materials may range from 0-99 percent (wt percent).

Because the density of the radiopaque additives is typically very high where the radiopaque materials are distributed throughout the matrix of bioabsorbable materials, dispersion techniques are preferably employed to distribute the radiopaque additives throughout the bioabsorbable materials as desired. Such techniques include high shear mixing, surfactant and lubricant additions, viscosity control, surface modification of the additive, and other particle size, shape and distribution techniques. In this regard, it is noted that the radiopaque materials may be either uniformly distributed throughout the bioabsorbable materials of the device, or may be concentrated in sections of the device so as to appear as markers similar to as described above.

Polymer tubes, for example, may be prepared such that radiopaque materials may be either fully dispersed in it or preferentially dispersed only at certain locations. For example, a high concentration of the radiopaque agent may be only at the ends of the tubes. Different processes may be used to form these markers. One option is to drill or laser cut tiny holes or channels at the ends of tubes and filling it with the agent and coating it with the polymer. Another option is to prepare tubes and then attach the tubular marker bands at the ends by methods such as ultrasonic welding, localized heating at the boundary, gluing them with polymer solution or fusing them when the tube and marker bands are not fully dry when prepared from solvent based processes. The advantage for these approaches is that marker bands may be added or attached at any location on the tubes that are prepared without radiopaque agents.

The local delivery of therapeutic agent/therapeutic agent combinations may be utilized to treat a wide variety of conditions utilizing any number of medical devices, or to enhance the function and/or life of the device. For example, intraocular lenses, placed to restore vision after cataract surgery is often compromised by the formation of a secondary cataract. The latter is often a result of cellular overgrowth on the lens surface and can be potentially minimized by combining a drug or drugs with the device. Other medical devices which often fail due to tissue in-growth or accumulation of proteinaceous material in, on and around the device, such as shunts for hydrocephalus, dialysis grafts, colostomy bag attachment devices, ear drainage tubes, leads for pace makers and implantable defibrillators can also benefit from the device-drug combination approach. Devices which serve to improve the structure and function of tissue or organ may also show benefits when combined with the appropriate agent or agents. For example, improved osteointegration of orthopedic devices to enhance stabilization of the implanted device could potentially be achieved by combining it with agents such as bone-morphogenic protein. Similarly other surgical devices, sutures, staples, anastomosis devices, vertebral disks, bone pins, suture anchors, hemostatic barriers, clamps, screws, plates, clips, vascular implants, tissue adhesives and sealants, tissue scaffolds, various types of dressings, bone substitutes, intraluminal devices, including stents, stent-grafts and other devices for repairing aneurysims, and vascular supports could also provide enhanced patient benefit using this drug-device combination approach. Perivascular wraps may be particularly advantageous, alone or in combination with other medical devices. The perivascular wraps may supply additional drugs to a treatment site. Essentially, any other type of medical device may be coated in some fashion with a drug or drug combination, which enhances treatment over use of the singular use of the device or pharmaceutical agent.

In addition to various medical devices, the coatings on these devices may be used to deliver therapeutic and pharmaceutic agents including, all the compounds described above and anti-proliferative agents, anti-thrombogenic agents, anti-restenotic agents, anti-infective agents, anti-viral agents, anti-bacterial agents, anti-fungal agents, anti-inflammatory agents, cytostatic agents, cytotoxic agents, immunosuppressive agents, anti-microbial agents, anti-calcification agents, anti-encrustation agents, statins, hormones, anti-cancer agents, anti-coagulants, anti-migrating agents and tissue growth promoting agents.

As described herein, various drugs or agents may be incorporated into the medical device by a number of mechanisms, including blending it with the polymeric materials or affixing it to the surface of the device. Different drugs may be utilized as therapeutic agents, including sirolimus, or rapamycin, heparin, everolimus, tacrolimus, paclitaxel, cladribine as well as classes of drugs such as statins. These drugs and/or agents may be hydrophilic, hydrophobic, lipophilic and/or lipophobic.

The local delivery of drug/drug combinations from a stent has the following advantages; namely, the prevention of vessel recoil and remodeling through the scaffolding action of the stent and the prevention of multiple components of neointimal hyperplasia or restenosis as well as a reduction in inflammation and thrombosis. This local administration of drugs, agents or compounds to stented coronary arteries may also have additional therapeutic benefit. For example, higher tissue concentrations of the drugs, agents or compounds may be achieved utilizing local delivery, rather than systemic administration. In addition, reduced systemic toxicity may be achieved utilizing local delivery rather than systemic administration while maintaining higher tissue concentrations. Also in utilizing local delivery from a stent rather than systemic administration, a single procedure may suffice with better patient compliance. An additional benefit of combination drug, agent, and/or compound therapy may be to reduce the dose of each of the therapeutic drugs, agents or compounds, thereby limiting their toxicity, while still achieving a reduction in restenosis, inflammation and thrombosis. Local stent-based therapy is therefore a means of improving the therapeutic ratio (efficacy/toxicity) of anti-restenosis, anti-inflammatory, anti-thrombotic drugs, agents or compounds.

Rapamycin is a macroyclic triene antibiotic produced by streptomyces hygroscopicus as disclosed in U.S. Pat. No. 3,929,992. It has been found that rapamycin inhibits the proliferation of vascular smooth muscle cells in vivo. Accordingly, rapamycin may be utilized in treating intimal smooth muscle cell hyperplasia, restenosis and vascular occlusion in a mammal, particularly following either biologically or mechanically mediated vascular injury, or under conditions that would predispose a mammal to suffering such a vascular injury. Rapamycin functions to inhibit smooth muscle cell proliferation and does not interfere with the re-endothelialization of the vessel walls.

Rapamycin functions to inhibit smooth muscle cell proliferation through a number of mechanisms. In addition; rapamycin reduces the other effects caused by vascular injury, for example, inflammation. The mechanisms of action and various functions of rapamycin are described in detail below. Rapamycin as used throughout this application shall include rapamycin, rapamycin analogs, derivatives and congeners that bind FKBP12 and possess the same pharmacologic properties as rapamycin, as described in detail below.

Rapamycin reduces vascular hyperplasia by antagonizing smooth muscle proliferation in response to mitogenic signals that are released during angioplasty. Inhibition of growth factor and cytokine mediated smooth muscle proliferation at the late G1 phase of the cell cycle is believed to be the dominant mechanism of action of rapamycin. However, rapamycin is also known to prevent T-cell proliferation and differentiation when administered systemically. This is the basis for its immunosuppressive activity and its ability to prevent graft rejection.

The molecular events that are responsible for the actions of rapamycin, a known anti-proliferative, which acts to reduce the magnitude and duration of neointimal hyperplasia, are still being elucidated. It is known, however, that rapamycin enters cells and binds to a high-affinity cytosolic protein called FKBP12. The complex of rapamycin and FKPB12 in turn binds to and inhibits a phosphoinositide (PI)-3 kinase called the “mammalian Target of Rapamycin” or TOR. TOR is a protein kinase that plays a key role in mediating the downstream signaling events associated with mitogenic growth factors and cytokines in smooth muscle cells and T lymphocytes. These events include phosphorylation of p27, phosphorylation of p70 s6 kinase and phosphorylation of 4BP-1, an important regulator of protein translation.

It is recognized that rapamycin reduces restenosis by inhibiting neointimal hyperplasia. However, there is evidence that rapamycin may also inhibit the other major component of restenosis, namely, negative remodeling. Remodeling is a process whose mechanism is not clearly understood but which results in shrinkage of the external elastic lamina and reduction in lumenal area over time, generally a period of approximately three to six months in humans.

Negative or constrictive vascular remodeling may be quantified angiographically as the percent diameter stenosis at the lesion site where there is no stent to obstruct the process. If late lumen loss is abolished in-lesion, it may be inferred that negative remodeling has been inhibited. Another method of determining the degree of remodeling involves measuring in-lesion external elastic lamina area using intravascular ultrasound (IVUS). Intravascular ultrasound is a technique that can image the external elastic lamina as well as the vascular lumen. Changes in the external elastic lamina proximal and distal to the stent from the post-procedural timepoint to four-month and twelve-month follow-ups are reflective of remodeling changes.

Evidence that rapamycin exerts an effect on remodeling comes from human implant studies with rapamycin coated stents showing a very low degree of restenosis in-lesion as well as in-stent. In-lesion parameters are usually measured approximately five millimeters on either side of the stent i.e. proximal and distal. Since the stent is not present to control remodeling in these zones which are still affected by balloon expansion, it may be inferred that rapamycin is preventing vascular remodeling.

The data in Table 1 below illustrate that in-lesion percent diameter stenosis remains low in the rapamycin treated groups, even at twelve months. Accordingly, these results support the hypothesis that rapamycin reduces remodeling.

TABLE 1.0
Angiographic In-Lesion Percent Diameter Stenosis
(%, mean ± SD and “n =”) In Patients Who Received a
Rapamycin-Coated Stent

| Coating | Post | 4-6 month | 12 month |
|---|---|---|---|
| Group | Placement | Follow Up | Follow Up |
| Brazil | 10.6 ± 5.7 (30) | 13.6 ± 8.6 (30) | 22.3 ± 7.2 (15) |
| Netherlands | 14.7 ± 8.8 | 22.4 ± 6.4 | |

Additional evidence supporting a reduction in negative remodeling with rapamycin comes from intravascular ultrasound data that was obtained from a first-in-man clinical program as illustrated in Table 2 below.

TABLE 2.0
Matched IVUS data in Patients Who Received a
Rapamycin-Coated Stent

| | | 4-Month | 12-Month |
|---|---|---|---|
| | | Follow-Up | Follow-Up |
| IVUS Parameter | Post (n =) | (n =) | (n =) |
| Mean proximal vessel area | 16.53 ± 3.53 | 16.31 ± 4.36 | 13.96 ± 2.26 |
| (mm²) | (27) | (28) | (13) |
| Mean distal vessel area | 13.12 ± 3.68 | 13.53 ± 4.17 | 12.49 ± 3.25 |
| (mm²) | (26) | (26) | (14) |

The data illustrated that there is minimal loss of vessel area proximally or distally which indicates that inhibition of negative remodeling has occurred in vessels treated with rapamycin-coated stents.

Other than the stent itself, there have been no effective solutions to the problem of vascular remodeling. Accordingly, rapamycin may represent a biological approach to controlling the vascular remodeling phenomenon.

It may be hypothesized that rapamycin acts to reduce negative remodeling in several ways. By specifically blocking the proliferation of fibroblasts in the vascular wall in response to injury, rapamycin may reduce the formation of vascular scar tissue. Rapamycin may also affect the translation of key proteins involved in collagen formation or metabolism.

Rapamycin used in this context includes rapamycin and all analogs, derivatives and congeners that bind FKBP12 and possess the same pharmacologic properties as rapamycin.

In a preferred embodiment, the rapamycin is delivered by a local delivery device to control negative remodeling of an arterial segment after balloon angioplasty as a means of reducing or preventing restenosis. While any delivery device may be utilized, it is preferred that the delivery device comprises a stent that includes a coating or sheath which elutes or releases rapamycin. The delivery system for such a device may comprise a local infusion catheter that delivers rapamycin at a rate controlled by the administrator. In other embodiments, an injection need may be utilized.

Rapamycin may also be delivered systemically using an oral dosage form or a chronic injectable depot form or a patch to deliver rapamycin for a period ranging from about seven to forty-five days to achieve vascular tissue levels that are sufficient to inhibit negative remodeling. Such treatment is to be used to reduce or prevent restenosis when administered several days prior to elective angioplasty with or without a stent.

Data generated in porcine and rabbit models show that the release of rapamycin into the vascular wall from a nonerodible polymeric stent coating in a range of doses (35-430 ug/15-18 mm coronary stent) produces a peak fifty to fifty-five percent reduction in neointimal hyperplasia as set forth in Table 3 below. This reduction, which is maximal at about twenty-eight to thirty days, is typically not sustained in the range of ninety to one hundred eighty days in the porcine model as set forth in Table 4 below.

TABLE 3.0
Animal Studies with Rapamycin-coated stents.
Values are mean ± Standard Error of Mean

| | | | | | Neointimal Area | % Change From | |
|---|---|---|---|---|---|---|---|
| Study | Duration | Stent¹ | Rapamycin | N | (mm²) | Polyme | Metal |
| Porcine | | | | | | | |
| 98009 | 14 days | Metal | | 8 | 2.04 ± 0.17 | | |
| | | 1X + rapamycin | 153 μg | 8 | 1.66 ± 0.17* | −42% | −19% |
| | | 1X + TC300 + rapamycin | 155 μg | 8 | 1.51 ± 0.19* | −47% | −26% |
| 99005 | 28 days | Metal | | 10 | 2.29 ± 0.21 | | |
| | | | | 9 | 3.91 ± 0.60** | | |
| | | 1X + TC30 + rapamycin | 130 μg | 8 | 2.81 ± 0.34 | | +23% |
| | | 1X + TC100 + rapamycin | 120 μg | 9 | 2.62 ± 0.21 | | +14% |
| 99006 | 28 days | Metal | | 12 | 4.57 ± 0.46 | | |
| | | EVA/BMA 3X | | 12 | 5.02 ± 0.62 | | +10% |
| | | 1X + rapamycin | 125 μg | 11 | 2.84 ± 0.31* ** | −43% | −38% |
| | | 3X + rapamycin | 430 μg | 12 | 3.06 ± 0.17* ** | −39% | −33% |
| | | 3X + rapamycin | 157 μg | 12 | 2.77 ± 0.41* ** | −45% | −39% |
| 99011 | 28 days | Metal | | 11 | 3.09 ± 0.27 | | |
| | | | | 11 | 4.52 ± 0.37 | | |
| | | 1X + rapamycin | 189 μg | 14 | 3.05 ± 0.35 | | −1% |
| | | 3X + rapamycin/dex | 182/363 μg | 14 | 2.72 ± 0.71 | | −12% |
| 99021 | 60 days | Metal | | 12 | 2.14 ± 0.25 | | |
| | | 1X + rapamycin | 181 μg | 12 | 2.95 ± 0.38 | | +38% |
| 99034 | 28 days | Metal | | 8 | 5.24 ± 0.58 | | |
| | | 1X + rapamycin | 186 μg | 8 | 2.47 ± 0.33** | | −53% |
| | | 3X + rapamycin/dex | 185/369 μg | 6 | 2.42 ± 0.64** | | −54% |
| 20001 | 28 days | Metal | | 6 | 1.81 ± 0.09 | | |
| | | 1X + rapamycin | 172 μg | 5 | 1.66 ± 0.44 | | −8% |
| 20007 | | | | | | | |
| | 30 days | Metal | | 9 | 2.94 ± 0.43 | | |
| | | 1XTC + rapamycin | 155 μg | 10 | 1.40 ± 0.11* | | −52%* |
| Rabbit | | | | | | | |
| 99019 | 28 days | Metal | | 8 | 1.20 ± 0.07 | | |
| | | EVA/BMA 1X | | 10 | 1.26 ± 0.16 | | +5% |
| | | 1X + rapamycin | 64 μg | 9 | 0.92 ± 0.14 | −27% | −23% |
| | | 1X + rapamycin | 196 μg | 10 | 0.66 ± 0.12* ** | −48% | −45% |
| 99020 | 28 days | Metal | | 12 | 1.18 ± 0.10 | | |
| | | EVA/BMA 1X + rapamycin | 197 μg | 8 | 0.81 ± 0.16 | | −32% |

¹Stent nomenclature: EVA/BMA 1X, 2X, and 3X signifies approx. 500 μg, 1000 μg, and 1500 μg total mass (polymer + drug), respectively. TC, top coat of 30 μg, 100 μg, or 300 μg drug-free BMA; Biphasic: 2 × 1X layers of rapamycin in EVA/BMA spearated by a 100 μg drug-free BMA layer.
²0.25 mg/kg/d × 14 d preceeded by a loading dose of 0.5 mg/kg/d × 3 d prior to stent implantation.
*p < 0.05 from EVA/BMA control.
**p < 0.05 from Metal;
^{#}Inflammation score: (0 = essentially no intimal involvement; 1 = <25% intima involved; 2 = ≧25% intima involved; 3 = >50% intima involved).

TABLE 4.0
180 day Porcine Study with Rapamycin-coated stents.
Values are mean ± Standard Error of Mean

| | | | | | Neointimal | % Change From | | Inflammation |
|---|---|---|---|---|---|---|---|---|
| Study | Duration | Stent¹ | Rapamycin | N | Area (mm²) | Polyme | Metal | Score # |
| 20007 | 3 days | Metal | | 10 | 0.38 ± 0.06 | | | 1.05 ± 0.06 |
| (ETP-2-002233-P) | | 1XTC + rapamycin | 155 μg | 10 | 0.29 ± 0.03 | | −24% | 1.08 ± 0.04 |
| | 30 days | Metal | | 9 | 2.94 ± 0.43 | | | 0.11 ± 0.08 |
| | | 1XTC + rapamycin | 155 μg | 10 | 1.40 ± 0.11* | | −52%* | 0.25 ± 0.10 |
| | 90 days | Metal | | 10 | 3.45 ± 0.34 | | | 0.20 ± 0.08 |
| | | 1XTC + rapamycin | 155 μg | 10 | 3.03 ± 0.29 | | −12% | 0.80 ± 0.23 |
| | | 1X + rapamycin | 171 μg | 10 | 2.86 ± 0.35 | | −17% | 0.60 ± 0.23 |
| | 180 days | Metal | | 10 | 3.65 ± 0.39 | | | 0.65 ± 0.21 |
| | | 1XTC + rapamycin | 155 μg | 10 | 3.34 ± 0.31 | | −8% | 1.50 ± 0.34 |
| | | 1X + rapamycin | 171 μg | 10 | 3.87 ± 0.28 | | +6% | 1.68 ± 0.37 |

The release of rapamycin into the vascular wall of a human from a nonerodible polymeric stent coating provides superior results with respect to the magnitude and duration of the reduction in neointimal hyperplasia within the stent as compared to the vascular walls of animals as set forth above.

Humans implanted with a rapamycin coated stent comprising rapamycin in the same dose range as studied in animal models using the same polymeric matrix, as described above, reveal a much more profound reduction in neointimal hyperplasia than observed in animal models, based on the magnitude and duration of reduction in neointima. The human clinical response to rapamycin reveals essentially total abolition of neointimal hyperplasia inside the stent using both angiographic and intravascular ultrasound measurements. These results are sustained for at least one year as set forth in Table 5 below.

TABLE 5.0
Patients Treated (N = 45 patients) with a Rapamycin-coated Stent

| | Sirolimus FIM | 95% |
|---|---|---|
| | (N = 45 Patients, 45 | Confidence |
| Effectiveness Measures | Lesions) | Limit |
| Procedure Success (QCA) | 100.0% (45/45) | [92.1%, 100.0%] |
| 4-month In-Stent Diameter Stenosis (%) | | |
| Mean ± SD (N) | 4.8% ± 6.1% (30) | [2.6%, 7.0%] |
| Range (min, max) | (−8.2%, 14.9%) | |
| 6-month In-Stent Diameter Stenosis (%) | | |
| Mean ± SD (N) | 8.9% ± 7.6% (13) | [4.8%, 13.0%] |
| Range (min, max) | (−2.9%, 20.4%) | |
| 12-month In-Stent Diameter Stenosis | | |
| (%) | | |
| Mean ± SD (N) | 8.9% ± 6.1% (15) | [5.8%, 12.0%] |
| Range (min, max) | (−3.0%, 22.0%) | |
| 4-month In-Stent Late Loss (mm) | | |
| Mean ± SD (N) | 0.00 ± 0.29 (30) | [−0.10, 0.10] |
| Range (min, max) | (−0.51, 0.45) | |
| 6-month In-Stent Late Loss (mm) | | |
| Mean ± SD (N) | 0.25 ± 0.27 (13) | [0.10, 0.39] |
| Range (min, max) | (−0.51, 0.91) | |
| 12-month In-Stent Late Loss (mm) | | |
| Mean ± SD (N) | 0.11 ± 0.36 (15) | [−0.08, 0.29] |
| Range (min, max) | (−0.51, 0.82) | |
| 4-month Obstruction Volume (%) (IVUS) | | |
| Mean ± SD (N) | 10.48% ± 2.78% (28) | [9.45%, 11.51%] |
| Range (min, max) | (4.60%, 16.35%) | |
| 6-month Obstruction Volume (%) (IVUS) | | |
| Mean ± SD (N) | 7.22% ± 4.60% (13) | [4.72%, 9.72%], |
| Range (min, max) | (3.82%, 19.88%) | |
| 12-month Obstruction Volume (%) | | |
| (IVUS) | | |
| Mean ± SD (N) | 2.11% ± 5.28% (15) | [0.00%, 4.78%], |
| Range (min, max) | (0.00%, 19.89%) | |
| 6-month Target Lesion Revascularization (TLR) | 0.0% (0/30) | [0.0%, 9.5%] |
| 12-month Target Lesion Revascularization | 0.0% (0/15) | [0.0%, 18.1%] |
| (TLR) | | |

QCA = Quantitative Coronary Angiography
SD = Standard Deviation
IVUS = Intravascular Ultrasound

Rapamycin produces an unexpected benefit in humans when delivered from a stent by causing a profound reduction in in-stent neointimal hyperplasia that is sustained for at least one year. The magnitude and duration of this benefit in humans is not predicted from animal model data. Rapamycin used in this context includes rapamycin and all analogs, derivatives and congeners that bind FKBP12 and possess the same pharmacologic properties as rapamycin.

These results may be due to a number of factors. For example, the greater effectiveness of rapamycin in humans is due to greater sensitivity of its mechanism(s) of action toward the pathophysiology of human vascular lesions compared to the pathophysiology of animal models of angioplasty. In addition, the combination of the dose applied to the stent and the polymer coating that controls the release of the drug is important in the effectiveness of the drug.

As stated above, rapamycin reduces vascular hyperplasia by antagonizing smooth muscle proliferation in response to mitogenic signals that are released during angioplasty injury. Also, it is known that rapamycin prevents T-cell proliferation and differentiation when administered systemically. It has also been determined that rapamycin exerts a local inflammatory effect in the vessel wall when administered from a stent in low doses for a sustained period of time (approximately two to six weeks). The local anti-inflammatory benefit is profound and unexpected. In combination with the smooth muscle anti-proliferative effect, this dual mode of action of rapamycin may be responsible for its exceptional efficacy.

Accordingly, rapamycin delivered from a local device platform, reduces neointimal hyperplasia by a combination of anti-inflammatory and smooth muscle anti-proliferative effects. Rapamycin used in this context means rapamycin and all rapamycin analogs, derivatives and congeners that bind FKBP12 and possess the same pharmacologic properties as rapamycin. Local device platforms include stent coatings, stent sheaths, grafts and local drug infusion catheters or porous balloons or any other suitable means for the in situ or local delivery of drugs, agents or compounds.

The anti-inflammatory effect of rapamycin is evident in data from an experiment, illustrated in Table 6, in which rapamycin delivered from a stent was compared with dexamethasone delivered from a stent. Dexamethasone, a potent steroidal anti-inflammatory agent, was used as a reference standard. Although dexamethasone is able to reduce inflammation scores, rapamycin is far more effective than dexamethasone in reducing inflammation scores. In addition, rapamycin significantly reduces neointimal hyperplasia, unlike dexamethasone.

TABLE 6.0

| Group | | | | |
|---|---|---|---|---|
| Rapamycin | | Neointimal Area | % Area | Inflammation |
| Rap | N = | (mm²) | Stenosis | Score |
| Uncoated | 8 | 5.24 ± 1.65 | 54 ± 19 | 0.97 ± 1.00 |
| Dexamethasone | 8 | 4.31 ± 3.02 | 45 ± 31 | 0.39 ± 0.24 |
| (Dex) | | | | |
| Rapamycin | 7 | 2.47 ± 0.94* | 26 ± 10* | 0.13 ± 0.19* |
| (Rap) | | | | |
| Rap + Dex | 6 | 2.42 ± 1.58* | 26 ± 18* | 0.17 ± 0.30* |

*= significance level P < 0.05

The drugs, agents or compounds described herein may be utilized in combination with any number of medical devices, and in particular, with implantable medical devices such as stents and stent-grafts. Other devices such as vena cava filters and anastomosis devices may be used with coatings having drugs, agents or compounds therein or the devices themselves may be fabricated with polymeric materials that have the drugs contained therein. Any of the stents or other medical devices described herein may be utilized for local or regional drug delivery. Balloon expandable stents may be utilized in any number of vessels or conduits, and are particularly well suited for use in coronary arteries. Self-expanding stents, on the other hand, are particularly well suited for use in vessels where crush recovery is a critical factor, for example, in the carotid artery.

Any of the above-described medical devices may be utilized for the local delivery of drugs, agents and/or compounds to other areas, not immediately around the device itself. In order to avoid the potential complications associated with systemic drug delivery, the medical devices of the present invention may be utilized to deliver therapeutic agents to areas adjacent to the medical device. For example, a rapamycin coated stent may deliver the rapamycin to the tissues surrounding the stent as well as areas upstream of the stent and downstream of the stent (regional delivery). The degree of tissue penetration depends on a number of factors, including the drug, agent or compound, the concentrations of the drug and the release rate of the agent. The same holds true for coated anastomosis devices.

The amount of drugs or other agents incorporated within the drug delivery device according to the systems and methods of the present invention may range from about 0 to 99 percent (percent weight of the device). The drugs or other agents may be incorporated into the device in different ways. For example, the drugs or other agents may be coated onto the device after the device has been formed, wherein the coating is comprised of bioabsorbable polymers into which the drugs or other agents are incorporated. Alternately, the drugs or other agents may be incorporated into the matrix of bioabsorbable materials comprising the device. The drugs or agents incorporated into the matrix of bioabsorbable polymers may be in an amount the same as, or different than, the amount of drugs or agents provided in the coating techniques discussed earlier if desired. These various techniques of incorporating drugs or other agents into, or onto, the drug delivery device may also be combined to optimize performance of the device, and to help control the release of the drugs or other agents from the device.

Where the drug or agent is incorporated into the matrix of bioabsorbable polymers comprising the device, for example, the drug or agent will release by diffusion and during degradation of the device. The amount of drug or agent released by diffusion will tend to release for a longer period of time than occurs using coating techniques, and may often more effectively treat local and diffuse lesions or conditions thereof. For regional drug or agent delivery such diffusion release of the drugs or agents is effective as well. Polymer compositions and their diffusion and absorption characteristics will control drug elution profile for these devices. The drug release kinetics will be controlled by drug diffusion and polymer absorption. Initially, most of the drug will be released by diffusion from the device surfaces and bulk and will then gradually transition to drug release due to polymer absorption. There may be other factors that will also control drug release. If the polymer composition is from the same monomer units (e.g., lactide; glycolide), then the diffusion and absorption characteristics will be more uniform compared to polymers prepared from mixed monomers. Also, if there are layers of different polymers with different drug in each layer, then there will be more controlled release of drug from each layer. There is a possibility of drug present in the device until the polymer fully absorbs thus providing drug release throughout the device life cycle.

The drug delivery device according to the systems and methods of the present invention preferably retains its mechanical integrity during the active drug delivery phase of the device. After drug delivery is achieved, the structure of the device ideally disappears as a result of the bioabsorption of the materials comprising the device. The bioabsorbable materials comprising the drug delivery device are preferably biocompatible with the tissue in which the device is implanted such that tissue interaction with the device is minimized even after the device is deployed within the patient. Minimal inflammation of the tissue in which the device is deployed is likewise preferred even as degradation of the bioabsorbable materials of the device occurs. In order to provide multiple drug therapy, enriched or encapsulated drug particles or capsules may be incorporated in the polymer matrix. Some of these actives may provide different therapeutic benefits such as anti-inflammatory, anti-thrombotic; etc.

In accordance with another exemplary embodiment, the stents described herein, whether constructed from metals or polymers, may be utilized as therapeutic agents or drug delivery devices wherein the drug is affixed to the surface of the device. The metallic stents may be coated with a biostable or bioabsorbable polymer or combinations thereof with the therapeutic agents incorporated therein. Typical material properties for coatings include flexibility, ductility, tackiness, durability, adhesion and cohesion. Biostable and bioabsorbable polymers that exhibit these desired properties include methacrylates, polyurethanes, silicones, poly(vinyl acetate), poly(vinyl alcohol), ethylene vinyl alcohol, poly(vinylidene fluoride), poly(lactic acid), poly(glycolic acid), poly(caprolactone), poly(trimethylene carbonate), poly(dioxanone), polyorthoester, polyanhydrides, polyphosphoester, polyaminoacids as well as their copolymers and blends thereof.

In addition to the incorporation of therapeutic agents, the surface coatings may also include other additives such as radiopaque constituents, chemical stabilizers for both the coating and/or the therapeutic agent, radioactive agents, tracing agents such as radioisotopes such as tritium (i.e. heavy water) and ferromagnetic particles, and mechanical modifiers such as ceramic microspheres as will be described in greater detail subsequently. Alternatively, entrapped gaps may be created between the surface of the device and the coating and/or within the coating itself. Examples of these gaps include air as well as other gases and the absence of matter (i.e. vacuum environment). These entrapped gaps may be created utilizing any number of known techniques such as the injection of microencapsulated gaseous matter.

As described above, different drugs may be utilized as therapeutic agents, including sirolimus, heparin, everolimus, tacrolimus, paclitaxel, cladribine as well as classes of drugs such as statins. These drugs and/or agents may be hydrophilic, hydrophobic, lipophilic and/or lipophobic. The type of agent will play a role in determining the type of polymer. The amount of the drug in the coating may be varied depending on a number of factors including, the storage capacity of the coating, the drug, the concentration of the drug, the elution rate of the drug as well as a number of additional factors. The amount of drug may vary from substantially zero percent to substantially one hundred percent. Typical ranges may be from about less than one percent to about forty percent or higher. Drug distribution in the coating may be varied. The one or more drugs may be distributed in a single layer, multiple layers, single layer with a diffusion barer or any combination thereof.

Different solvents may be used to dissolve the drug/polymer blend to prepare the coating formulations. Some of the solvents may be good or poor solvents based on the desired drug elution profile, drug morphology and drug stability.

There are several ways to coat the stents that are disclosed in the prior art. Some of the commonly used methods include spray coating; dip coating; electrostatic coating; fluidized bed coating; and supercritical fluid coatings.

Some of the processes and modifications described herein that may be used will eliminate the need for polymer to hold the drug on the stent. Stent surfaces may be modified to increase the surface area in order to increase drug content and tissue-device interactions. Nanotechnology may be applied to create self-assembled nanomaterials that can contain tissue specific drug containing nanoparticles. Microstructures may be formed on surfaces by microetching in which these nanoparticles may be incorporated. The microstructures may be formed by methods such as laser micromachining, lithography, chemical vapor deposition and chemical etching. Microstructures may be added to the stent surface by vapor deposition techniques. Microstructures have also been fabricated on polymers and metals by leveraging the evolution of micro electro-mechanical systems (MEMS) and microfluidics. Examples of nanomaterials include carbon nanotubes and nanoparticles formed by sol-gel technology. Therapeutic agents may be chemically or physically attached or deposited directly on these surfaces. Combination of these surface modifications may allow drug release at a desired rate. A top-coat of a polymer may be applied to control the initial burst due to immediate exposure of drug in the absence of polymer coating.

As described above, polymer stents may contain therapeutic agents as a coating, e.g. a surface modification. Alternatively, the therapeutic agents may be incorporated into the stent structure, e.g. a bulk modification that may not require a coating. For stents prepared from biostable and/or bioabsorbable polymers, the coating, if used, could be either biostable or bioabsorbable. However, as stated above, no coating may be necessary because the device itself is fabricated from a delivery depot. This embodiment offers a number of advantages. For example, higher concentrations of the therapeutic agent or agents may be achievable such as about >50 percent by weight. In addition, with higher concentrations of therapeutic agent or agents, regional drug delivery (>5 mm) is achievable for greater durations of time. This can treat different lesions such as diffused lesions, bifurcated lesions, small and tortuous vessels, and vulnerable plaque. Since these drug loaded stents or other devices have very low deployment pressures (3 to 12 atmospheres), it will not injure the diseased vessels. These drug-loaded stents can be delivered by different delivery systems such balloon expandable; self-expandable or balloon assist self-expanding systems.

In yet another alternate embodiment, the intentional incorporation of ceramics and/or glasses into the base material may be utilized in order to modify its physical properties. Typically, the intentional incorporation of ceramics and/or glasses would be into polymeric materials for use in medical applications. Examples of biostable and/or bioabsorbable ceramics or/or glasses include hydroxyapatite, tricalcium phosphate, magnesia, alumina, zirconia, yittrium tetragonal polycrystalline zirconia, amorphous silicon, amorphous calcium and amorphous phosphorous oxides. Although numerous technologies may be used, biostable glasses may be formed using industrially relevant sol-gel methods. Sol-gel technology is a solution process for fabricating ceramic and glass hybrids. Typically, the sol-gel process involves the transition of a system from a mostly colloidal liquid (sol) into a gel.

The sterilization process of the present invention is particularly adapted to the challenges of sterilizing drug coated medical devices. Specifically, the sterilization process is designed to remove all biological contaminants without affecting the drug, agent or compound or the polymeric material comprising the device or the coating.

In accordance with one exemplary embodiment, a low temperature sterilization method may be utilized to sterilize the devices of the present invention. The method comprises the steps of positioning at least one packaged, drug coated or drug containing medical device in a sterilization chamber, creating a vacuum in the sterilization chamber, increasing and maintaining the temperature in the sterilization chamber in the range from about twenty-five degrees C. to about forty degrees C. and the relative humidity in the sterilization chamber in the range from about forty percent to about eighty-five percent for a first predetermined period, injecting a sterilization agent at a predetermined concentration into the sterilization chamber and maintaining the temperature in the sterilization chamber in the range from about twenty-five degrees C. to about forty degrees C. and the relative humidity in the range from about forty percent to about eighty-five percent for a second predetermined period, and removing the sterilization agent from the sterilization chamber through a plurality of vacuum and nitrogen washes over a third predetermined period, the temperature in the sterilization chamber being maintained at a temperature in the range from about thirty degrees C. to about forty degrees C.

In accordance with another exemplary embodiment, a low temperature sterilization method may be utilized to sterilize the devices of the present invention. The method comprising the steps of loading the at least one packaged, drug coated medical device in a preconditioning chamber, the preconditioning chamber being maintained at a first predetermined temperature and a first predetermined relative humidity for a first predetermined time period, positioning at least one packaged, drug coated medical device in a sterilization chamber creating a vacuum in the sterilization chamber increasing and maintaining the temperature in the sterilization chamber in the range from about twenty-five degrees C. to about forty degrees C. and the relative humidity in the sterilization chamber in the range from about forty percent to about eighty-five percent for a first predetermined period injecting a sterilization agent at a predetermined concentration into the sterilization chamber and maintaining the temperature in the sterilization chamber in the range from about twenty-five degrees C. to about forty degrees C. and the relative humidity in the range from about forty percent to about eighty-five percent for a second predetermined period, and removing the sterilization agent from the sterilization chamber through a plurality of vacuum and nitrogen washes over a third predetermined period, the temperature in the sterilization chamber being maintained at a temperature in the range from about thirty degrees C. to about forty degrees C.

In each embodiment described above, the sterilization or sterilizing agent may comprise ethylene oxide or any other suitable agent. The nitrogen washes, which serve to remove the ethylene oxide may be replaced with other suitable gases, including any of the noble gases.

Other sterilization methods may also be used, such gamma and electron beam radiations. In these methods the dosage should be low so that drug in the devices is not adversely affected. The dosage may range from about one to four mrad and more preferably below 2 mrad. Radiation sterilized polymers will absorb relatively faster than ethylene oxide sterilized polymers.

Although shown and described is what is believed to be the most practical and preferred embodiments, it is apparent that departures from specific designs and methods described and shown will suggest themselves to those skilled in the art and may be used without departing from the spirit and scope of the invention. The present invention is not restricted to the particular constructions described and illustrated, but should be constructed to cohere with all modifications that may fall within the scope for the appended claims.

What is claimed is:

1. An implantable intraluminal medical device comprising:
a structure formed from at least one polymer; and
at least one therapeutic agent dispersed throughout the at least one polymer in a concentration of up to thirty percent.

2. The implantable intraluminal medical device according to claim 1, wherein the at least one polymer comprises a blend of one or more polymers.

3. The implantable intraluminal medical device according to claim 1, wherein the at least one polymer comprises a blend of at least one polymer and at least one plasticizer.

4. The implantable intraluminal medical device according to claim 1, wherein the structure comprises a stent.

5. The implantable intraluminal medical device according to claim 1, wherein the at least one polymer comprises bioabsorbable polymers.

6. The implantable intraluminal medical device according to claim 1, wherein the at least one polymer comprises non-bioabsorbable polymers.

7. The implantable intraluminal medical device according to claim 5, wherein the bioabsorbable polymer comprises poly(alpha hydroxy esters).

8. The implantable intraluminal medical device according to claim 6, wherein the non-bioabsorbable polymer comprises polyurethane.

9. The implantable intraluminal medical device according to claim 1, wherein the structure comprises a covered stent.

10. The implantable intraluminal medical device according to claim 1, wherein the at least one therapeutic agent comprises anti-proliferative agents.

11. The implantable intraluminal medical device according to claim, 1 wherein the at least one therapeutic agent comprises anti-thrombogenic agents.

12. The implantable intraluminal medical device according to claim 1, wherein the at least one therapeutic agent comprises anti-restenotic agents.

13. The implantable intraluminal medical device according to claim 1, wherein the at least one therapeutic agent comprises anti-infective agents.

14. The implantable intraluminal medical device according to claim 1, wherein the at least one therapeutic agent comprises anti-viral agents.

15. The implantable intraluminal medical device according to claim 1, wherein the at least one therapeutic agent comprises anti-bacterial agents.

16. The implantable intraluminal medical device according to claim 1, wherein the at least one therapeutic agent comprises anti-fungal agents.

17. The implantable intraluminal medical device according to claim 1, wherein the at least one therapeutic agent comprises anti-inflammatory agents.

18. The implantable intraluminal medical device according to claim 1, wherein the at least one therapeutic agent comprises cytostatic agents.

19. The implantable intraluminal medical device according to claim 1, wherein the at least one therapeutic agent comprises cytotoxic agents.

20. The implantable intraluminal medical device according to claim 1, wherein the at least one therapeutic agent comprises immunosuppressive agents.

21. The implantable intraluminal medical device according to claim 1, wherein the at least one therapeutic agent comprises anti-microbial agents.

22. The implantable intraluminal medical device according to claim 1, wherein the at least one therapeutic agent comprises anti-calcification agents.

23. The implantable intraluminal medical device according to claim 1, wherein the at least one therapeutic agent comprises anti-encrustation agents.

24. The implantable intraluminal medical device according to claim 1, wherein the at least one therapeutic agent comprises statins.

25. The implantable intraluminal medical device according to claim 1, wherein the at least one therapeutic agent comprises hormones.

26. The implantable intraluminal medical device according to claim 1, wherein the at least one therapeutic agent comprises anti-cancer agents.

27. The implantable intraluminal medical device according to claim 1, wherein the at least one therapeutic agent comprises anti-coagulants.

28. The implantable intraluminal medical device according to claim 1, wherein the at least one therapeutic agent comprises anti-migratory agents.

29. The implantable intraluminal medical device according to claim 1, wherein the at least one therapeutic agent comprises tissue growth promoting agents.

30. The implantable intraluminal medical device according to claim 1, wherein the structure comprises a heparin coated stent.

31. An implantable intraluminal medical device comprising:
a structure formed from a first material; and
a coating layer affixed to the first material, the coating layer including at least one therapeutic agent dispersed throughout a polymeric material in a concentration of up to thirty percent.

32. The implantable intraluminal medical device according to claim 31, wherein the structure comprises a stent.

33. The implantable intraluminal medical device according to claim 31, wherein the at least one polymer comprises bioabsorbable polymers.

34. The implantable intraluminal medical device according to claim 31, wherein the at least one polymer comprises non-bioabsorbable polymers.

35. The implantable intraluminal medical device according to claim 33, wherein the bioabsorbable polymer comprises poly(alpha hydroxy esters).

36. The implantable intraluminal medical device according to claim 34, wherein the non-bioabsorbable polymer comprises polyurethane.

37. The implantable intraluminal medical device according to claim 31, wherein the at least one therapeutic agent comprises anti-proliferative agents.

38. The implantable intraluminal medical device according to claim 31, wherein the at least one therapeutic agent comprises anti-thrombogenic agents.

39. The implantable intraluminal medical device according to claim 31, wherein the at least one therapeutic agent comprises anti-restenotic agents.

40. The implantable intraluminal medical device according to claim 31, wherein the at least one therapeutic agent comprises anti-infective agents.

41. The implantable intraluminal medical device according to claim 31, wherein the at least one therapeutic agent comprises anti-viral agents.

42. The implantable intraluminal medical device according to claim 31, wherein the at least one therapeutic agent comprises anti-bacterial agents.

43. The implantable intraluminal medical device according to claim 31, wherein the at least one therapeutic agent comprises anti-fungal agents.

44. The implantable intraluminal medical device according to claim 31, wherein the at least one therapeutic agent comprises anti-inflammatory agents.

45. The implantable intraluminal medical device according to claim 31, wherein the at least one therapeutic agent comprises cytostatic agents.

46. The implantable intraluminal medical device according to claim 31, wherein the at least one therapeutic agent comprises cytotoxic agents.

47. The implantable intraluminal medical device according to claim 31, wherein the at least one therapeutic agent comprises immunosuppressive agents.

48. The implantable intraluminal medical device according to claim 31, wherein the at least one therapeutic agent comprises anti-microbial agents.

49. The implantable intraluminal medical device according to claim 31, wherein the at least one therapeutic agent comprises anti-calcification agents.

50. The implantable intraluminal medical device according to claim 31, wherein the at least one therapeutic agent comprises anti-encrustation agents.

51. The implantable intraluminal medical device according to claim 31, wherein the at least one therapeutic agent comprises statins.

52. The implantable intraluminal medical device according to claim 31, wherein the at least one therapeutic agent comprises hormones.

53. The implantable intraluminal medical device according to claim 31, wherein the at least one therapeutic agent comprises anti-cancer agents.

54. The implantable intraluminal medical device according to claim 31, wherein the at least one therapeutic agent comprises anti-coagulants.

55. The implantable intraluminal medical device according to claim 31, wherein the at least one therapeutic agent comprises anti-migratory agents.

56. The implantable intraluminal medical device according to claim 31, wherein the at least one therapeutic agent comprises tissue growth promoting agents.

57. The implantable intraluminal medical device according to claim 31, wherein the structure comprises a covered stent.

58. The implantable intraluminal medical device according to claim 31, wherein the structure comprises a heparin coated stent.

59. An implantable intraluminal medical device comprising:
a structure formed from at least one polymer; and
at least one radiopaque agent dispersed throughout the at least one polymer in a concentration of up to thirty percent.

60. The implantable intraluminal medical device according to claim 59, wherein the structure comprises a stent.

61. The implantable intraluminal medical device according to claim 59, wherein the at least one polymer comprises bioabsorbable polymers.

62. The implantable intraluminal medical device according to claim 59, wherein the at least one polymer comprises non-bioabsorbable polymers.

63. The implantable intraluminal medical device according to claim 61, wherein the bioabsorbable polymer comprises poly(alpha hydroxy esters).

64. The implantable intraluminal medical device according to claim 62, wherein the non-bioabsorbable polymer comprises polyurethane.

65. The implantable intraluminal medical device according to claim 59, wherein the at least one radiopaque agent comprises barium sulfate.

66. The implantable intraluminal medical device according to claim 59, wherein the structure comprises a covered stent.

67. The implantable intraluminal medical device according to claim 59, wherein the at least one radiopaque agent comprises gold.

68. The implantable intraluminal medical device according to claim 59, wherein the at least one radiopaque agent comprises tantalum.

69. The implantable intraluminal medical device according to claim 59, wherein the at least one radiopaque agent comprises platinum.

70. The implantable intraluminal medical device according to claim 59, wherein the at least one radiopaque agent comprises palladium.

71. The implantable intraluminal medical device according to claim 59, wherein the at least one radiopaque agent comprises tungsten.

72. The implantable intraluminal medical device according to claim 59, wherein the at least one radiopaque agent is chemically attached to the polymer.

73. The implantable intraluminal medical device according to claim 59, wherein the at least one radiopaque agent is physically mixed with the polymer.

74. An implantable intraluminal medical device comprising:
a structure formed from a first material; and
a coating layer affixed to the first material, the coating layer including at least one radiopaque agent dispersed throughout a polymeric material in a concentration of up to thirty percent.

75. The implantable intraluminal medical device according to claim 74, wherein the structure comprises a stent.

76. The implantable intraluminal medical device according to claim 74, wherein the at least one polymer comprises bioabsorbable polymers.

77. The implantable intraluminal medical device according to claim 74 wherein the at least one polymer comprises non-bioabsorbable polymers.

78. The implantable intraluminal medical device according to claim 76, wherein the bioabsorbable polymer comprises poly(alpha hydroxy esters).

79. The implantable intraluminal medical device according to claim 77, wherein the non-bioabsorbable polymer comprises polyurethane.

80. The implantable intraluminal medical device according to claim 74, wherein the at least one radiopaque agent comprises barium sulfate.

81. The implantable intraluminal medical device according to claim 74, wherein structure comprises a covered stent.

82. An implantable intraluminal medical device comprising:
a structure formed from at least one polymer;
at least one radiopaque agent; and
at least one therapeutic agent dispersed throughout the at least one polymer in a concentration of up to thirty percent.

83. An implantable intraluminal medical device comprising:
a structure formed from at least one polymer;
at least one therapeutic agent; and
at least one radiopaque agent dispersed throughout the at least one polymer in a concentration of up to thirty percent.

84. An implantable intraluminal medical device comprising:
a structure formed from a first material; and
a coating layer affixed to the first material, the coating layer including at least one therapeutic agent and at least one radiopaque agent dispersed throughout a polymeric material in a concentration of up to thirty percent.

85. An implantable intraluminal medical device comprising:
a structure formed from a first material; and
a coating layer affixed to the first material, the coating layer including at least one radiopaque agent and at least one therapeutic agent dispersed throughout a polymeric material in a concentration of up to thirty percent.

86. An implantable intraluminal medical device comprising:
a structure having a proximal end and a distal end, the structure being formed from at least one polymer; and
at least one therapeutic agent dispersed for elution of the at least one therapeutic agent from the at least one polymer, wherein the dispersion of the at least one therapeutic agent allows for elution of the at least one therapeutic agent to a distance of greater than about five mm proximal from the proximal end and to a distance of greater than about five mm distal from the distal end.

87. The implantable intraluminal medical device according to claim 86, wherein the concentration of the at least one therapeutic agent in the tissue at a distance of greater than five mm proximal or distal is equal to or greater than a therapeutic dosage.

88. The implantable intraluminal medical device according to claim 86, further comprising a radiopaque agent.

89. An implantable intraluminal medical device comprising:
a structure having a proximal and a distal end, the structure being formed from a first material; and
a coating layer affixed to the first material, the coating layer including at least one therapeutic agent distributed for elution of the at least one therapeutic agent in the at least one polymer, wherein the distribution of the at least one therapeutic agent allows for elution of the at least one therapeutic agent to a distance of greater than about five mm proximal from the proximal end and to a distance of greater than about five mm distal from the distal end.

90. The implantable intraluminal medical device according to claim 89, further comprising a radiopaque agent.

91. An implantable intraluminal medical device comprising:
a structure being formed from at least one polymer; and
at least one therapeutic agent distributed for elution of the at least one therapeutic agent in the at least one polymer, wherein the distribution of the at least one therapeutic agent allows for regional delivery.

92. The implantable intraluminal medical device according to claim 91, wherein the structure comprises a bifurcated stent.

93. The implantable intraluminal medical device according to claim 91, wherein the structure comprises a stent.

94. The implantable intraluminal medical device according to claim 91, wherein the structure comprises a vascular filter.

95. The implantable intraluminal medical device according to claim 91, wherein the structure comprises an aneurismal repair device.

96. The implantable intraluminal medical device according to claim 91, wherein the structure comprises devices for treating diffuse arterial lesions.

97. The implantable intraluminal medical device according to claim 91, wherein the structure comprises devices for treating superficial femoral artery disease.

98. The implantable intraluminal medical device according to claim 91, wherein the structure comprises devices for treating below the knee arterial disease.

99. The implantable intraluminal medical device according to claim 91, wherein the structure comprises venous valves.

100. The implantable intraluminal medical device according to claim 91, wherein the structure comprises heart valves.

101. The implantable intraluminal medical device according to claim 91, wherein regional delivery includes elution of the at least one therapeutic agent upstream of the structure.

102. The implantable intraluminal medical device according to claim 91, wherein regional delivery includes elution of the at least one therapeutic agent downstream of the structure.

103. The implantable intraluminal medical device according to claim 91, wherein regional delivery includes elution of the at least one therapeutic agent to an adjacent vessel proximate to the structure.

104. The implantable intraluminal medical device according to claim 91, wherein regional delivery includes elution of the at least one therapeutic agent to an organ proximate to the structure.

105. The implantable intraluminal medical device according to claim 91, further comprising a radiopaque agent.

106. The implantable intraluminal medical device according to claim 91, wherein the structure comprises devices for treating smaller vessels.

107. The implantable intraluminal medical device according to claim 91, wherein the structure comprises devices for treating tapered vessels.

108. The implantable intraluminal medical device according to claim 91, wherein the structure comprises devices for treating tortuous vessels.

109. An implantable intraluminal medical device comprising:
a structure being formed from at least one polymer; and
at least one therapeutic agent dispersed throughout the at least one polymer, wherein concentration of the dispersion provides for the controlled elution of the at least one therapeutic agent for greater than about one day.

110. The implantable intraluminal medical device according to claim 109, further comprising a radiopaque agent.

111. An implantable intraluminal medical device comprising:
a structure being formed from at least one polymer; and
at least one therapeutic agent dispersed throughout the at least one polymer, wherein concentration of the dispersion provides for the controlled elution of the at least one therapeutic agent for greater than about sixty days.

112. An implantable intraluminal medical device comprising:
a non-fibrous structure formed from at least one polymer; and
at least one therapeutic agent dispersed throughout the at least one polymer in a concentration of up to thirty percent.

113. The implantable intraluminal medical device according to claim 112, further comprising a radiopaque agent.

114. A method for forming an implantable medical device comprising the steps of:
creating a matrix from at least one biocompatible polymer;
dispersing at least one therapeutic agent in the matrix to create a raw material, the therapeutic agent having a degradation temperature;
heating the raw material to a maximum solvent processing temperature in the range from about one degree Celsius less than the degradation temperature to about eighty degrees Celsius less than the degradation temperature of the at least one therapeutic agent; and
forming the heated raw material into an implantable medical device.

115. The method according to claim 114, wherein the step of forming the heated raw material into an implantable medical device comprises forming tubular structures by solvent casting.

116. The method according to claim 115, wherein the step of forming tubular structures by solvent coating further comprises deposition of the raw material on a rotating mandrel.

117. The method according to claim 115, further comprising forming the tubular structure into an implantable medical device utilizing an excimer laser processing.

118. The method according to claim 114, wherein the step of forming the heated raw material into an implantable medical device comprises forming tubular structures by supercritical fluid processing.

119. The method according to claim 114, wherein the step of forming the heated raw material into an implantable medical device comprises forming tubular structures by extrusion process.

120. The method according to claim 119, wherein the step of forming tubular structures by extrusion process comprises heating the raw material to a temperature in the range from about fifty degrees C. to about ninety degrees C.

121. The method according to claim 119, further comprising forming the tubular structure into an implantable medical device utilizing an excimer laser processing.

122. The method according to claim 114, wherein the step of forming the heated raw material into an implantable medical device comprises forming tubular structures by three dimensional printing processing.

123. The method according to claim 114, wherein the step of forming the heated raw material into an implantable medical device comprises forming film structures by solvent casting.

124. The method according to claim 123, further comprising forming the film structure into an implantable medical device utilizing an excimer laser processing.

125. The method according to claim 114, wherein the step of forming the heated raw material into an implantable medical device comprises forming film structures by supercritical fluid processing.

126. The method according to claim 114, wherein the step of forming the heated raw material into an implantable medical device comprises forming film structures by extrusion process.

127. The method according to claim 114, wherein the step of forming the heated raw material into an implantable medical device comprises forming film structures by three dimensional printing processing.

128. A method for forming an implantable medical device comprising the steps of:
creating a matrix from at least one biocompatible polymer;
dispersing at least one therapeutic agent in the matrix to create a raw material, the therapeutic agent having a degradation temperature;
heating the raw material to a maximum melt processing temperature in the range from about one degree Celsius less than the degradation temperature to about sixty degrees Celsius less than the degradation temperature of the at least one therapeutic agent; and
forming the heated raw material into an implantable medical device.

129. The method according to claim 128, wherein the step of forming the heated raw material into an implantable medical device comprises forming tubular structures by supercritical fluid processing.

130. The method according to claim 128, wherein the step of forming the heated raw material into an implantable medical device comprises forming tubular structures by extrusion process.

131. The method according to claim 130, further comprising forming the tubular structure into an implantable medical device utilizing an excimer laser processing.

132. The method according to claim 128, wherein the step of forming the heated raw material into an implantable medical device comprises forming tubular structures by three dimensional printing processing.

133. The method according to claim 128, wherein the step of forming the heated raw material into an implantable medical device comprises forming film structures by supercritical fluid processing.

134. The method according to claim 128, wherein the step of forming the heated raw material into an implantable medical device comprises forming film structures by extrusion process.

135. The method according to claim 128, wherein the step of forming the heated raw material into an implantable medical device comprises forming film structures by three dimensional printing processing.

136. The method according to claim 128, wherein the step of forming the heated raw material into an implantable medical device comprises forming tubular structures by molding processing.

137. The method according to claim 136, further comprising forming the tubular structure into an implantable medical device utilizing an excimer laser processing.

138. The method according to claim 128, wherein the step of forming the heated raw material in to an implantable medical device comprises molding the heated raw material directly in to a medical device.

139. The method according to claim 128, wherein the step of forming the heated raw material into an implantable medical device comprises forming film structures by molding process.

140. A method for forming an implantable intraluminal medical device comprising the steps of:
forming a raw material comprising at least one polymer into a medical device having a plurality of sections; and
dispersing at least one therapeutic agent into one or more of the plurality of sections to create predetermined elution profiles.

141. An implantable intraluminal medical device comprising:
a structure formed from at least one polymer, the at least one polymer configured to degrade for a period in the range from about one day to about three years; and
at least one therapeutic agent dispersed throughout the at least one polymer.

142. An implantable intraluminal medical device comprising:
a structure formed from a first material; and
a coating layer affixed to the first material, the coating layer including at least one therapeutic agent dispersed throughout a polymeric material being configured to degrade for a period in the range from about one day to about three years.

143. An implantable intraluminal medical device comprising:
a structure formed from at least one polymer, the at least one polymer configured to degrade for a period in the range from about one day to about three years; and
at least one radiopaque agent dispersed throughout the at least one polymer.

144. An implantable intraluminal medical device comprising:
a structure formed from a first material; and
a coating layer affixed to the first material, the coating layer including at least one radiopaque agent dispersed throughout a polymeric material being configured to degrade for a period in the range from about 1 day to 3 years.

145. A method for forming an implantable intraluminal medical device comprising the steps of:
forming a raw material comprising at least one polymer into a medical device having a plurality of sections; and
dispersing at least one radiopaque agent into one or more of the plurality of sections to create predetermined marker bands.

146. A method for forming an implantable intraluminal medical device comprising the steps of:
forming a raw material comprising at least one polymer into a medical device having a plurality of sections;
dispersing at least one therapeutic agent into one or more of the plurality of sections to create predetermined elution profiles; and
dispersing at least one radiopaque agent into one or more of the plurality of sections to create predetermined marker bands.

147. An implantable intraluminal medical device comprising:
a balloon expandable structure formed from at least one polymer; and
at least one therapeutic agent dispersed throughout the at least one polymer in a concentration of up to thirty percent.

148. An implantable intraluminal medical device comprising:
a balloon expandable structure formed from at least one polymer; and
at least one radiopaque agent dispersed throughout the at least one polymer in a concentration of up to thirty percent.

149. An implantable intraluminal medical device comprising:
a self expanding structure formed from at least one polymer; and
at least one therapeutic agent dispersed throughout the at least one polymer in a concentration of up to thirty percent.

150. An implantable intraluminal medical device comprising:
a self expanding structure formed from at least one polymer; and
at least one radiopaque agent dispersed throughout the at least one polymer in a concentration of up to thirty percent.

151. An implantable intraluminal medical device comprising:
a balloon expandable structure formed from at least one polymer;
at least one therapeutic agent dispersed throughout the at least one polymer in a concentration of up to thirty percent; and
at least one radiopaque agent dispersed throughout the at least one polymer in a concentration of up to thirty percent.

152. An implantable intraluminal medical device comprising:
a self expanding structure formed from at least one polymer;
at least one therapeutic agent dispersed throughout the at least one polymer in a concentration of up to thirty percent; and
at least one radiopaque agent dispersed throughout the at least one polymer in a concentration of up to thirty percent.

153. An implantable intraluminal medical device comprising:
a structure having interlocking segments formed from at least one polymer;
at least one therapeutic agent dispersed throughout the at least one polymer in a concentration of up to thirty percent; and
at least one radiopaque agent dispersed throughout the at least one polymer in a concentration of up to thirty percent.

154. An implantable intraluminal medical device comprising:
a structure having interlocking segments formed from at least one polymer; and at least one therapeutic agent dispersed throughout the at least one polymer in a concentration of up to thirty percent.

155. An implantable intraluminal medical device comprising:
a structure having interlocking segments formed from at least one polymer; and
at least one radiopaque agent dispersed throughout the at least one polymer in a concentration of up to thirty percent.

156. A method of deploying an intraluminal device comprising the steps of:
introducing a delivery system in to the vasculature at the treatment site;
expanding the intraluminal device utilizing a pressure ranging from about one atmosphere to about ten atmospheres.

157. The method according to claim 156, further comprising the step of holding the intraluminal device in the expanding state for a period of greater than about thirty seconds to less than ten minutes.

158. The method according to claim 157, further comprising the step of preconditioning the intraluminal device by exposing to a heat source.

159. A method of sterilizing a drug containing polymeric intraluminal device comprising the steps of:
placing the polymeric intraluminal device into a sterilization chamber;
introducing a sterilization agent into the chamber at a first temperature, a first pressure and a first humidity level for a first period of time, the first temperature not to exceed sixty degrees Celsius; and
removing the sterilization agent from the chamber by introducing an inert gas into the chamber at a second temperature, a second pressure and a second humidity level for a second period of time.

160. The method according to claim to 159, wherein the sterilizing agent is ethylene oxide and the inert gas is nitrogen.

161. The method according to claim to 159, wherein the inert gas is nitrogen.

162. A method for treating long lesions in the vasculature comprising the steps of:
positioning a structure having individual segments formed from at least one polymer and comprising at least one therapeutic agent dispersed throughout each of the individual segments in the at last one polymer in a concentration of up to thirty percent; and
expanding each of the individual segments to open and support the vasculature.

163. A method for treating long lesions in the vasculature comprising the steps of:
positioning a structure having individual scaffold segments formed from at least one polymer and comprising at least one therapeutic agent dispersed throughout each of the individual segments in the at last one polymer in a concentration of up to thirty percent; and
expanding each of the individual scaffold segments to open and support the vasculature.
